(19) United States
(12) Patent Application Publication
(10) Pub. No.: US 2025/0144445 A1
(43) Pub. Date: May 8, 2025

(54) Systems and Methods for Treating Catheter Infections

(71) Applicant: ABL Medical Inc., East Providence, RI (US)

(72) Inventors: Robert A. Rabiner, Dortmund (DE); Liane J. Rabiner, Dortmund (DE)

(73) Assignee: ABL Medical Inc., East Providence, RI (US)

(21) Appl. No.: 18/935,204

(22) Filed: Nov. 1, 2024

Related U.S. Application Data
(60) Provisional application No. 63/547,099, filed on Nov. 2, 2023.
(60) Provisional application No. 63/551,915, filed on Feb. 9, 2024.

(51) Int. Cl. A61N 5/06; A61M 25/00

(52) U.S. Cl. CPC A61N 5/0624; A61M 25/0017; A61M 25/0075; A61N 5/0601; A61M 2025/0019; A61M 2025/0076; A61M 2205/051; A61N 2005/0612; A61N 2005/063; A61N 2005/0644; A61N 2005/0663; A61N 2005/0668

(57) ABSTRACT

Systems and methods are provided for treating catheter infections. In some embodiments, a device is provided that includes a main body including a first leg configured to engage a catheter, a second leg configured to engage a drainage tube, and a third leg configured to receive one or more optical fibers, and a port coupled to the third leg. The port is configured to provide access for the one or more optical fibers and provide a seal relative to the one or more optical fibers. The one or more optical fibers are configured to disperse light energy in the catheter such that intensity of the light energy dispersed in the catheter is distributed evenly over a length of the one or more optical fibers in both longitudinal and circumferential directions.

### RELATED APPLICATIONS

This application claims the benefit of and priority to U.S. Provisional Application No. 63/551,915 filed Feb. 9, 2024, and U.S. Provisional Application No. 63/547,099 filed Nov. 2, 2023, and the contents of each of these applications are incorporated herein by reference in their entireties.

### FIELD

The embodiments disclosed herein relate to treatments for infections related to a catheter, and more particularly to anti-microbial blue light systems and methods for providing anti-microbial and/or anti-bacterial effects for catheter applications.

### BACKGROUND

Various catheters are used in a number of medical and surgical procedures. Depending on the type of catheter being used and the location of the body effected, it can be possible for an infection to occur related to the catheter use. These infections can be viral or bacterial in nature. Once a catheter infection has occurred, it can be difficult to treat depending on the location of the catheter. These infections can be common but if not addressed properly can cause severe health problems. It would be desirable to have an improved systems and methods for eliminating infections, bacteria, or other pathogens associated with catheter use.

### SUMMARY

The present disclosure is directed to systems, devices, and methods for providing treatment for catheter infections. In some embodiments, a device is provided that includes a main body including a first leg configured to engage a catheter, a second leg configured to engage a drainage tube, and a third leg configured to receive one or more optical fibers, and a port coupled to the third leg. The port is configured to provide access for the one or more optical fibers and provide a seal relative to the one or more optical fibers. The one or more optical fibers are configured to disperse light energy in the catheter such that intensity of the light energy dispersed in the catheter is distributed evenly over a length of the one or more optical fibers in both longitudinal and circumferential directions.

In some embodiments, the device further includes an anti-reflux valve configured to prevent fluid from flowing in a wrong direction out of the catheter.

In some embodiments, the device further includes a stop to hold the one or more optical fibers in position relative to the main body.

In some embodiments, the device further includes a compressive member configured to engage the catheter to increase a force of the seal around the catheter to increase a sterile barrier around the catheter. In some embodiments, the compressive member is configured to apply a circumferential force to engage the catheter with the main body.

In some embodiments, the device further includes a strain relief configured to support the one or more optical fibers for a distance to prevent the one or more optical fibers from deforming during use.

In some embodiments, the catheter is a Foley catheter.

In some embodiments, the one or more optical fibers are configured to disperse light energy to treat pathogens associated with urinary tract infection. In some embodiments, the one or more optical fibers include a cladding covering an outer surface thereof, and wherein at least a portion of the cladding of the one or more optical fibers is removed from an outer surface of the one or more optical fibers to achieve an even dispersion of the light energy. In some embodiments, the one or more optical fibers sized to pass through an inner lumen of the catheter and being configured to deliver light energy to provide an antimicrobial effect to the tissue.

In some embodiments, a system is provided that includes a catheter having an elongated shaft and an inner lumen therethrough, a main body including a first leg configured to engage the catheter, a second leg configured to engage a drainage tube, and a third leg configured to receive one or more optical fibers, and the one or more optical fibers sized to pass through the third leg of the main body into the inner lumen of the delivery catheter and being configured to deliver light energy to provide an antimicrobial effect to the tissue. The one or more optical fibers are configured to disperse the light energy such that an intensity of the light energy is distributed evenly over a length of the one or more optical fibers in both longitudinal and circumferential directions; wherein the antimicrobial effect of the light energy is configured to affect bacteria.

In some embodiments, the system further includes a port coupled to the third leg, the port being configured to provide access for the one or more optical fibers and provide a seal relative to the one or more optical fibers.

In some embodiments, the one or more optical fibers include a cladding covering an outer surface thereof, and wherein at least a portion of the cladding of the one or more optical fibers is removed from an outer surface of the one or more optical fibers to achieve an even dispersion of the light energy. In some embodiments, at least a portion of the cladding is removed to form a helical spiral along the length of the one or more optical fibers. In some embodiments, the helical spiral becomes increasingly tight as the helical spiral moves from a proximal end of the one or more optical fibers to a distal end of the one or more optical fibers to achieve an even light distribution over the length of the one or more optical fibers.

In some embodiments, the one or more optical fibers includes a diffusive membrane disposed on an outer surface thereof, the diffusive membrane configured to be applied to the outer surface of the one or more optical fibers to achieve the even light distribution over the length of the one or more optical fibers.

In some embodiments, the light energy has illumination wavelengths from about 400 nm to about 475 nm. In some embodiments, the light energy has illumination wavelengths from about 380 nm to about 500 nm. In some embodiments, the light energy has illumination wavelengths from about 405 nm to about 470 nm.

In some embodiments, a method is provided that includes engaging a catheter with a fitting, the fitting including a first leg configured to engage the catheter, a second leg configured to engage a drainage tube, and a third leg configured to receive one or more optical fibers; receiving, by the third leg of the fitting, the one or more optical fibers; passing the one or more optical fibers from the third leg of fitting into an inner lumen of the catheter; and delivering light energy from the one or more optical fibers to the catheter to provide an antimicrobial effect, the one or more optical fibers being configured to disperse the light energy such that an intensity of the light energy is distributed evenly over a length of the one or more optical fibers in both longitudinal and circumferential directions.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 depicts an exemplary embodiment of a universal fitting for use with a catheter;

FIG. 2 depicts an exemplary embodiment of a urinary fitting;

FIGS. 3A and 3B depict an exemplary embodiment of a retention arm and a compression ring for coupling a urinary fitting to a Foley catheter;

FIG. 4 depicts an exemplary embodiment of a main body of a urinary fitting;

FIG. 5 depicts an exemplary embodiment of a sealing valve;

FIG. 6A and FIG. 6B depict an exemplary embodiment of an inner seal;

FIG. 7 depicts an exemplary embodiment of an anti-reflux check valve;

FIG. 8 depicts an exemplary embodiment of a securing clamp;

FIG. 9 depicts an exemplary embodiment of a strain relief component;

FIG. 10A, FIG. 10B, and FIG. 10C depict exemplary embodiments of a Foley catheter for use with a fitting;

FIG. 10D depicts an exemplary embodiment of a Foley catheter for use with a fitting;

FIG. 11 illustrates a graph showing showing exemplary time-kill curves of Escherichia coli by blue light, using one, two, or three optical fibers, over a 6 hour period;

FIG. 12 illustrates a graph showing showing exemplary time-kill curves of Escherichia coli by blue light over 120 minutes;

FIG. 13 illustrates an exemplary graph showing power distribution over the length of the fiber;

FIG. 14A and FIG. 14B illustrates an exemplary reflective surface added to the distal tip of a light fiber;

FIG. 15A, FIG. 15B, FIG. 15C, and FIG. 15D show various views of an exemplary embodiment of a fiber with cladding removed in a spiral;

FIG. 16 shows an exemplary embodiment of a fiber with a portion of the cladding removed;

FIG. 17 shows that in some embodiments, the cladding is removed 360 degrees around the fiber; and in some embodiments, the cladding is only removed in a 180 degree orientation with the cut side outwards;

FIG. 18 illustrates an exemplary fiber having cladding that changes depth along the length of the fiber;

FIG. 19 is an exemplary test set up relating to experiments run for for a test strain of Escherichia coli AR11 (ESBL-prod. strain, 3MRGN);

FIGS. 20A, 20B, 20C, 20D, and 20E illustrate exemplary results for experiments run for the test strain of Escherichia coli AR11 (ESBL-prod. strain, 3MRGN);

FIGS. 21A, 21B, 21C, 21D, 21E, and 21F illustrate a test set up and results for experiments run for a test strain of Proteus mirabilis BLS-2;

FIGS. 22A, 22B, 22C, 22D, 22E, and 22F illustrate a test set up and results for experiments run for a test strain of Escherichia coli ATCC 35218 (TEM-1, non-ESBL);

FIGS. 23A, 23B, 23C, 23D, 23E, and 23F illustrate a test set up and results for experiments run for a test strain of Escherichia coli AR11 (ESBL-prod. strain, 3MRGN);

FIGS. 24A, 24B, 24C, 24D, 24E, and 24F illustrate a test set up and results for experiments run for a test strain of Proteus mirabilis BLS-2;

FIGS. 25A, 25B, 25C, 25D, 25E, and 25F illustrate a test set up and results for experiments run for a test strain of Proteus mirabilis BLS-2;

FIGS. 26A, 26B, 26C, 26D, 26E, and 26F illustrate a test set up and results for experiments run for a test strain of Escherichia coli ATCC 35218 (TEM-1, non-ESBL); and

FIGS. 27A, 27B, 27C, 27D, 27E, and 27F illustrate a test set up and results for experiments run for a test strain of Escherichia coli ATCC 25922.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Systems and methods for antimicrobial blue light photolysis (ABLP) for treatment of catheter infections (for example, urinary tract infections) and disorders are disclosed herein. An anti-microbial effect may also include a bactericidal effect or an anti-bacterial effect, among other things. In some embodiments, the ABLP systems and methods can be used in conjunction with procedures that utilize a catheter. For example, the fitting and ABLP can be used with urinary catheters, endotracheal catheters, dialysis catheters, cerebral spinal catheters, or other catheters. For example, light for providing an anti-microbial and/or anti-bacterial effect can be used in a variety of medical applications, including but not limited to surgery, interventional radiology, gynecology, infectious diseases, and wound care.

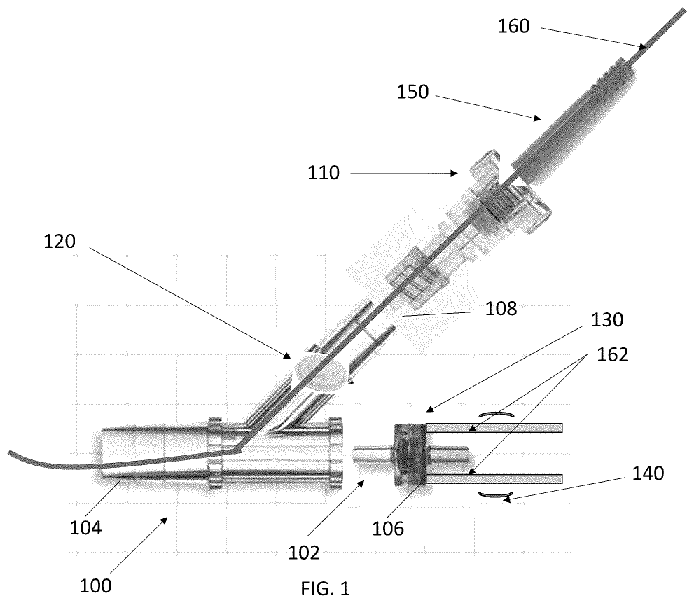

In some embodiments, a universal fitting 100 is provided to allow the application of a light fiber within a catheter, as shown in FIGS. 1-3. The fitting is configured to engage a catheter and receive one or more optical fibers such that the one or more optical fibers can pass through a portion of the fitting and into the catheter to delivery light to treat infection, bacteria, and/or other pathogens. In some embodiments, the fitting 100 can be used with any off-the-shelf catheter, making the fitting a universal component to be used with any closed catheter system. The fitting can be made from a variety of materials. In some embodiments, the fitting is plastic.

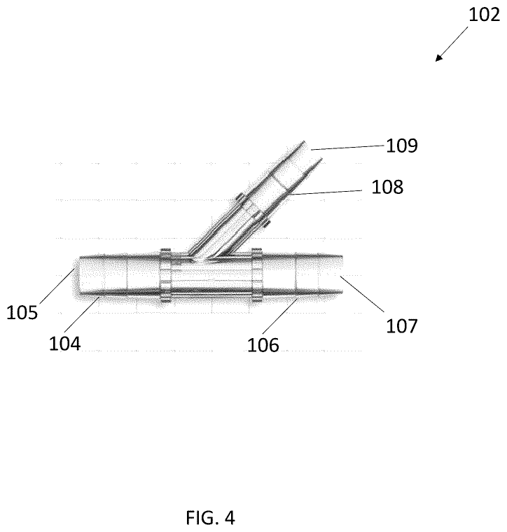

The fitting 100 comprises a main body 102, shown in more detail in FIG. 4, that includes a first leg 104 having a first opening 105 for receiving a catheter, a second leg 106 having a second opening 107 for receiving a drainage component 162, such as a drainage tube or bag, and a third leg 108 having a third opening 109 for receiving one or more optical fibers 160. The main body is configured such that the one or more optical fibers can pass through the third leg of the main body and into the catheter engaged with the first leg of the main body.

In some embodiments, the first and second legs 104, 106 are substantially in line with one another, and the third leg 108 can be positioned at an angle relative to the longitudinal axis of the first and second legs, as shown in FIG. 4. This allows the one or more optical fibers to pass through the third leg and into the catheter to deliver light energy to provide an anti-microbial and/or anti-bacterial effect.

The angle of the longitudinal axis of the third leg can vary relative to the axis of the first and second legs of the main body. For example, the third leg can be positioned at an angle of between 30 degrees to 45 degrees relative to the longitudinal axis of the first and second legs of the main body. For example, the third leg can be positioned at an angle of between 20 degrees to 30 degrees relative to the longitudinal axis of the first and second legs of the main body. For example, the third leg can be positioned at an angle of 30 degrees relative to the longitudinal axis of the first and second legs of the main body. This allows the one or more optical fibers to enter the catheter at a low angle. This can prevent overbending of the one or more optical fibers which can cause a decrease in light output. In addition, the angle is not so flat that the one or more optical fibers is difficult to thread into the catheter as there is no lead in angle.

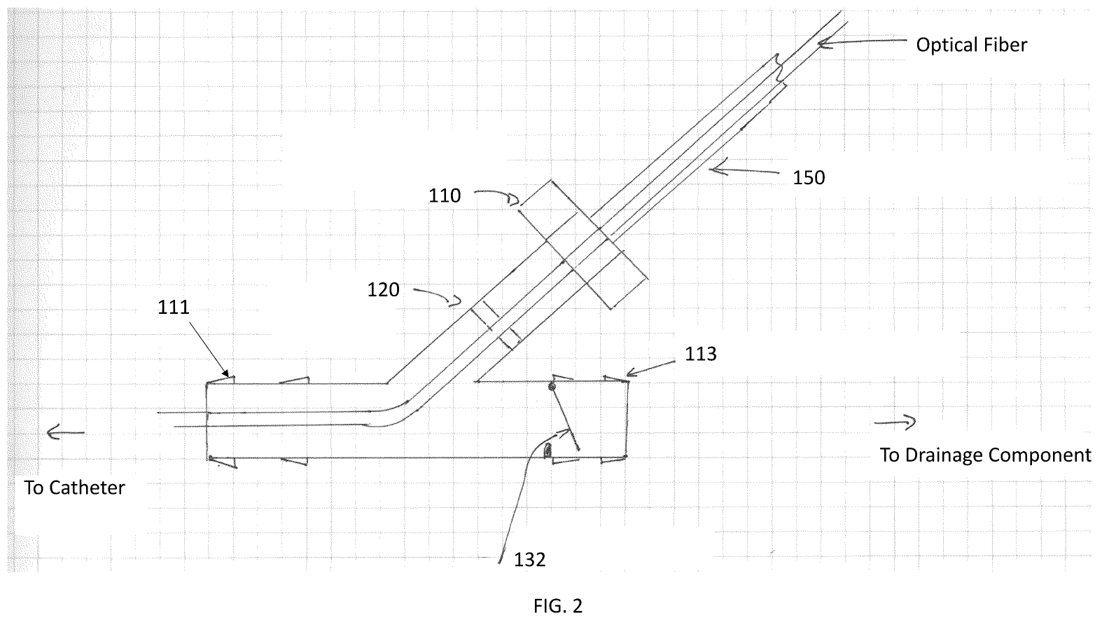

The fitting can include features to allow the catheter and drainage component to engage with the fitting or removably couple thereto. In some embodiments, as shown in FIG. 2, the first leg of the main body of the fitting can include a plurality of barbs 111 on an outer surface of the first leg, and the second leg of the main body can include a plurality of barbs 113 on an outer surface of the second leg. A proximal end of the catheter can be positioned over the barbs on the outer surface of the first leg to removably couple the catheter to the first leg. A distal end of the drainage component be positioned over the barbs on the outer surface of the second leg to removably couple the drain to the second leg.

In some embodiments, the first leg and/or the second leg can include an inner tube with an outer compressive clamp. Rather than using a press fit technique over the plurality of barbs on the outer surface of the legs, the catheter and/or drain can be configured to be positioned over a slip fitting. An outer circumferential compressive clip can be used to compress the catheter and/or drain to the first and second legs.

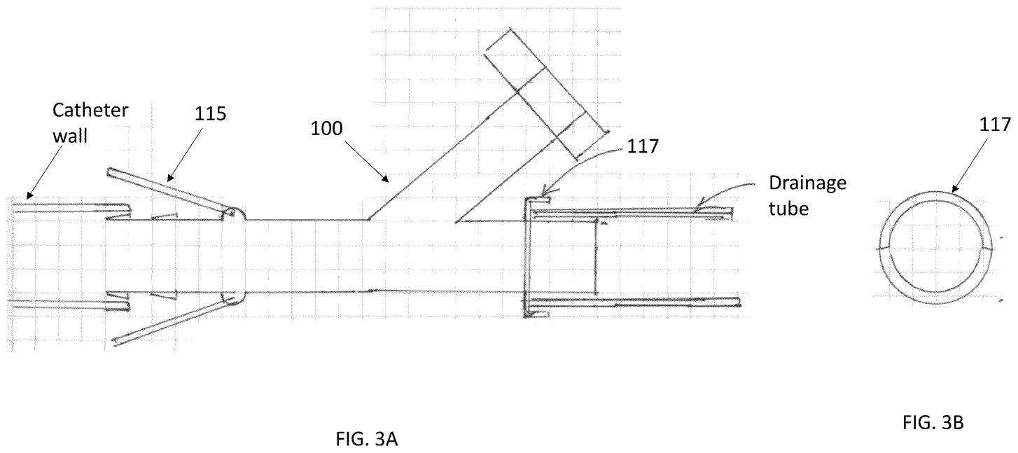

In some embodiments, the first leg includes a retention lock 115, as show in FIG. 3A, to hold the catheter in place relative to the first leg of the main body. The retention lock utilizes a friction fit to retain the catheter to removably couple the catheter to the fitting. A plurality of barbs or teeth are positioned on the outer surface of the fitting. At least a first and second retention arm are pivotally coupled to the outer surface of the first leg. The catheter can be positioned over the barbed end of the first leg and the at least first and second retention arms can be pivoted towards the catheter to lock the catheter to the outer surface of the first leg. In some embodiments, a compressive ring can be used to engage the catheter or the drainage component with the fitting. For example, a compressive ring can have a circular shape, with the top 180 degrees of the circumference and the bottom 180 degrees of a compressive ring 117 being configured to interlock, as shown in FIG. 3B, with one side being slightly larger than the other side so they fit within each other. In some embodiments, a compressive lock such as a compressing loop can be positioned over the first and second retention arms to lock the catheter to the first leg. It will be understood that either of the first and second legs can utilize any of the retention techniques to engage the fitting with the catheter and drainage component.

The location of the third leg relative to the first and second legs can also vary. In some embodiments, shown in FIG. 4, the third leg of the main body is positioned between sealing components of the first and second legs of the main body.

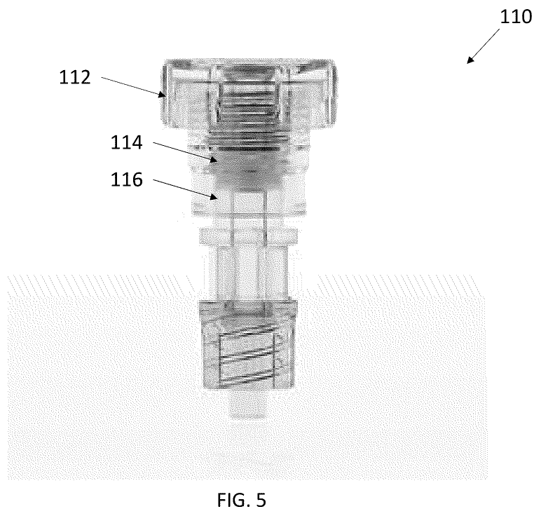

The fitting includes a port 110, or sealing valve, as shown in FIG. 5, that allows for the introduction of one or more optical fibers into the main body of the fitting through the third opening 108. The port 110 can be sized and shaped to be able to hold as many fibers as necessary for the required treatment. For example, the port can be sized to receive one, two, or three fibers.

The dimensions of the port can vary depending on the type of number of optical fibers being used. For example, the port can have a diameter of between 1.6 mm to 1.7 mm for receiving a single optical fiber. For example, the port can have a diameter of between 3.3 mm to 3.4 mm for receiving two optical fibers. For example, the port can have a diameter of between 3.6 mm-3.7 mm for receiving three optical fibers. For example, the port can have a diameter of between 3.9 mm-4.0 mm for receiving four optical fibers.

The port is configured to create a separate access location for one or more optical fibers such that a seal to created relative to the one or more optical fibers. The seal can also be adapted to prevent fluid egress from the port through which the one or more optical fibers is positioned, while still maintaining the ability to attach the Foley catheter to tubing on a drainage bag. The port can be self-sealing and/or lockable to hold the one or more optical fibers in position and/or prevent fluid ingress/egress. In some embodiments, the port 110 utilizes twist compression to provide the seal by twisting or rotation an outer portion 112 of the port to open and close the port.

In some embodiments, the port is formed from a smooth plastic. In some embodiments, the lumen that runs longitudinally through the port has a diameter that is configured to receive the one or more optical fibers. In some embodiments, the wall of the lumen is smooth to prevent any damage to the one or more optical fibers. For example, the inner lumen can be sized and include one or more recesses to hold one or more fibers. For example, the inner lumen can be sized to received two, three, or four fibers and their corresponding internal stabilizers.

The port can include a plurality of threads 114 on an inner surface to compress a flexible ring or gland 116, for example, made of silicon, positioned in the inner lumen of the port when the outer portion of the port is twisted or rotated. The ring allows the one or more optical fibers to pass through the port. The compression of the ring by the upper thread fills/seals the passage through the port and into the third leg of the main body of the fitting of any fluid. As the threaded screw are rotated, the silicon ring is configured to compress around the one or more optical fibers. This downwards compression can be used to force the silicone ring to be compressed inwards to seal the passage through the port.

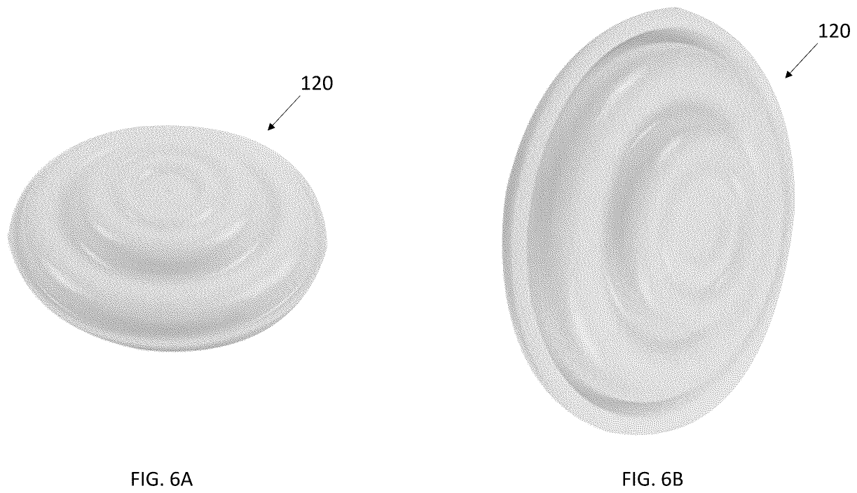

In some embodiments, the system utilizes dual seals to prevent loss of sterile integrity to the system. Thus, the port can be opened to insert/remove the light fiber without disturbing the sterile environment of the rest of the catheter. In some embodiments, the fitting includes the port (sealing valve) and a secondary valve, such as the inner seal 120 shown in FIG. 6A and FIG. 6B. In some embodiments, the inner seal 120 is positioned distal of the sealing valve 110. The combination of the port 110 and the inner seal 120 allows the one or more optical fibers to enter the main body of the fitting through the port 110, and then pass through the inner seal 120 to preserve the sterile environment inside the fitting. In some embodiments, the inner seal 120 is in the form of a flat, circular disk that provides a seal against the inner wall of the third leg of the fitting and allows the one or more optical fibers to pass therethrough.

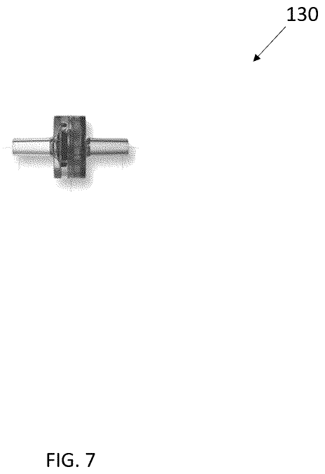

In some embodiments, the fitting can include an anti-reflux valve positioned in the second leg of the fitting which allows one or more fluids to be drained from though the fitting from the catheter. The anti-reflux valve can be configured to prevent fluid, such as urine, from flowing in the wrong direction out of the closed system of the catheter. The anti-reflux value of the universal fitting can thus prevent the accidental discharge of urine from the drainage bag to the Foley catheter, which can be one of the causes of CAUTI's. In some embodiments, as shown in FIG. 1 and in more detail in FIG. 7, an anti-reflex valve 130 can be in the form of a check valve for preventing fluid from leaving the drainage component and back into the main body of the fitting.

In some embodiment, as shown in FIG. 2, an anti-reflex valve can be in the form of a flap that moves relative to an inner wall of the second leg of the main body of the fitting. For example, the anti-reflex valve can be in the form of a duck bill having a one way fluid passage in the form of an elongated tube that has a thin wall that collapses unless fluid is running through it. Thus, fluid can flow downhill but not uphill through the tube. It can include a chamber that holds a ball or a flap 132 that floats in the chamber. When a fluid fills the chamber, the ball or flap floats up and allows the fluid flows out. Then the ball or flap closes. This fluid can flow only when there is fluid above a sealing system which causes the buoyancy of the flap to open. In some embodiments, there can be a plurality of chambers to ensure that there is no retrograde flow.

In some embodiments, the fitting includes a stop to hold the one or more optical fibers in place when positioned in the fitting. For example, the stop can be used to prevent the one or more optical fibers from either moving in or out of the fitting. In some embodiments, the stop can be in the form of a component attached to each fiber of the one or more optical fibers to provide physical interface to control movement of the fibers.

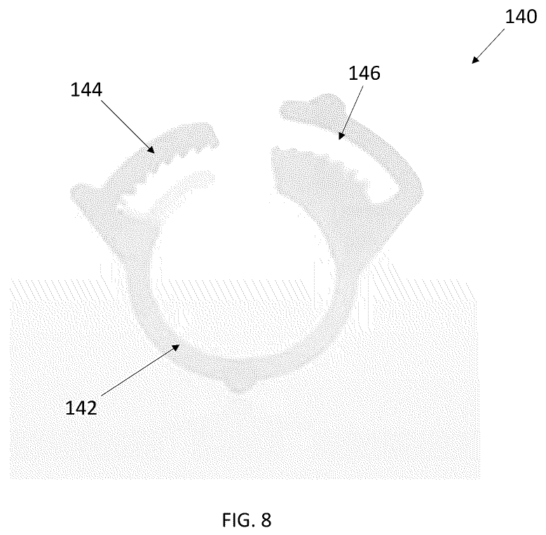

In some embodiments, the fitting includes a compressive member, or compressive clamp 140, as shown in FIG. 1 and FIG. 8, that is configured to grab onto the catheter. In some embodiments, the compressive member is positioned to couple to a standard entry port of the Foley catheter. The compressive member can be configured to removably clamp onto the drainage component, such as a drainage tubing line.

The compressive member 140 can be designed to apply circumferential force to hold the catheter to the fitting. The compression provided by the compressive member can increase the seal forces, and by extension increases the sterile barrier. The amount of force is enough to aid the coupling and/or increase the seal forces, but not enough to over-compress or crush the catheter or the inner fitting.

In some embodiments, as shown in FIG. 8, the compressive member has a circular body 142 such that the catheter can fit within the compressive member. The compressive member includes a split grip having a first tooth component 144 and a second tooth component 146 that are configured to engage with each other when the compressive member is compressed.

The compressive member provided additional security to the connection between the catheter and the fitting and is releasable to allow for an easy disconnection from either the fitting or the tubing. Thus, the compressive member can prevent disengagement of the catheter and the urinary drainage tubing, and/or can provide additional/incremental security against infections within the “sterile barrier” within the tubing.

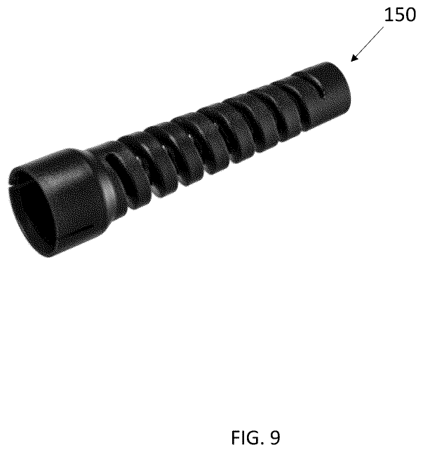

In some embodiments, the fitting includes a strain relief 150, as shown in FIG. 9, that is configured to support the light fiber for a distance. The strain relief can prevent the plastic optical fiber from being bent, crushed, or otherwise deformed during use. The strain relief of the universal fitting can be used to both protect the plastic light fiber and allow for mobility when not connected to the light source, as it is protected within the flexible strain relief. The length of the strain relief can vary. For example, the length of the strain relief can be 6 inches from a light box to a small forward section of the optical fiber, 4 feet from the light box to half the length of the fiber length, or 8 feet from the light box to the insertion point of the catheter fitting.

The dimensions of the fiber can vary depending on the dimensions of the Foley catheter. For example, the fiber dimensions can vary from 1 mm to 1.5 mm.

In use, a universal fitting is attached to a drainage lumen of a catheter, such as a Foley catheter. One or more light fibers is introduced to the central lumen of the catheter, the proximal end of the universal fitting is pressed on to the Foley catheter lumen, and the distal end of the fitting is attached to the drainage tubing.

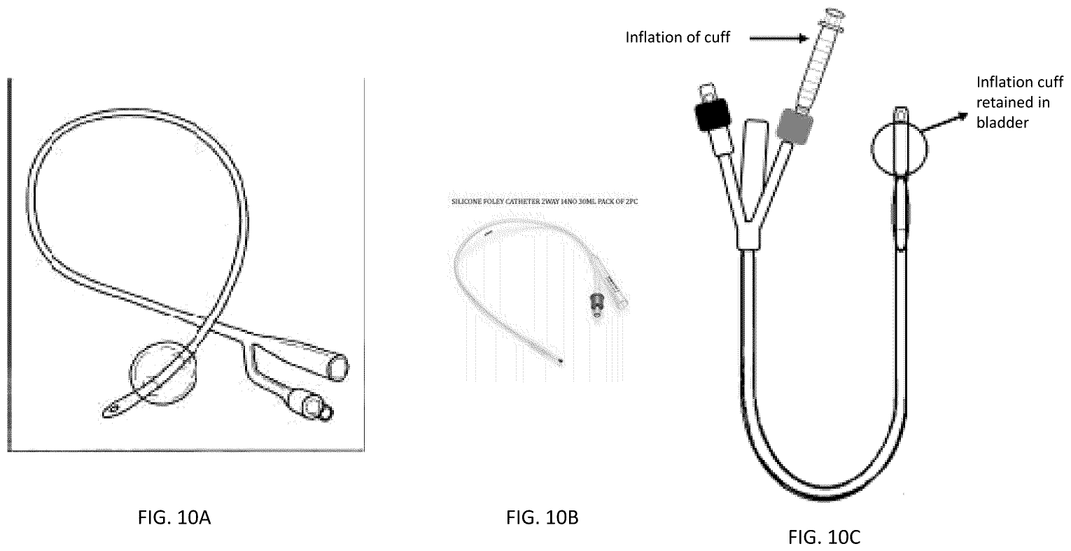

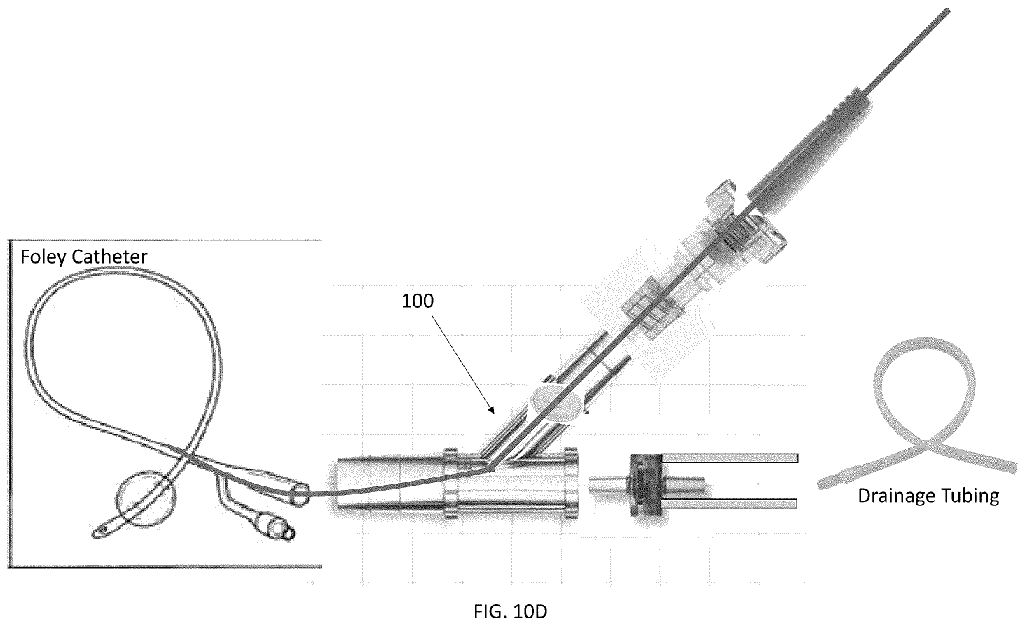

In some embodiments, the fitting 100 can be used with a Foley catheter, as shown in FIGS. 10A, 10B, 10C, and 10D. The fitting described herein is designed for the ease of use, with virtually no change in technique. While the light fiber simply is inserted within the existing lumen of the Foley, an accommodation fitting to allow the fiber to be delivered while connecting the drainage tube to the Foley has been provided. The universal fitting has been designed to ensure not only ease of use, not impede the current utilization standards of Foley catheter/drainage tubing use, but to provide incremental safety benefits to the Foley catheter, drainage tube connection as well.

Various infections/bacteria/pathogens can be targeted by a fiber inserted into a catheter. For example, in the case of a urinary (Foley) catheter, catheter associated urinary tract infections (CAUTI) are one of the most common healthcare associated infections. Each day the indwelling urinary catheter remains inserted, a patient has a 3%-7% increased risk of acquiring a catheter-associated urinary tract infection. UTI bacterial strains are relatively limited with Escherichia coli being the most common pathogen for both non-complicated and complicated UTI, making up 75% and 65% of infections, respectively followed by Klebsiella & Pseudomonas aeruginosa. The most common pathogens associated with CAUTI are gram-negative bacilli. The most frequent pathogens associated with CAUTI in hospitals reporting to National Healthcare Safety Network (NHSN) between 2015-2017 were Escherichia coli (34.3%), Klebsiella (14.2%) and Pseudomonas aeruginosa (12.8%). Frequencies of E. faecium and E. faecalis as CAUTI pathogens differed by location type, with E. faecium rarely identified in IRFs (1%) and E. faecalis commonly reported by oncology units (12%). Among CAUTIs, long-term acute-care hospitals had a significantly higher non-susceptibility rate than hospital wards for all pathogens. For example, 23% of Klebsiella were carbapenem-resistant Enterobacteriaceae in long-term acute-care hospitals.

Bacteria can enter the urinary tract in catheterized patients in three ways: (1) introduction of organisms into the bladder at the time of catheter insertion; (2) periurethral route; and (3) intraluminal route. The system described herein can treat all three variants of the initiation of the infection and can provide antimicrobial properties to the procedure.

For infections initiated within the bladder, one or more optical fibers can be introduced along the length of a Foley catheter and terminate within the small portion of the catheter residing within the bladder (for example, the catheter length beyond the retention balloon of the Foley). Testing has shown that even with only a single fiber, a bladder volume of 250 ml can be treated, and achieve a 3 log reduction in 4 hours.

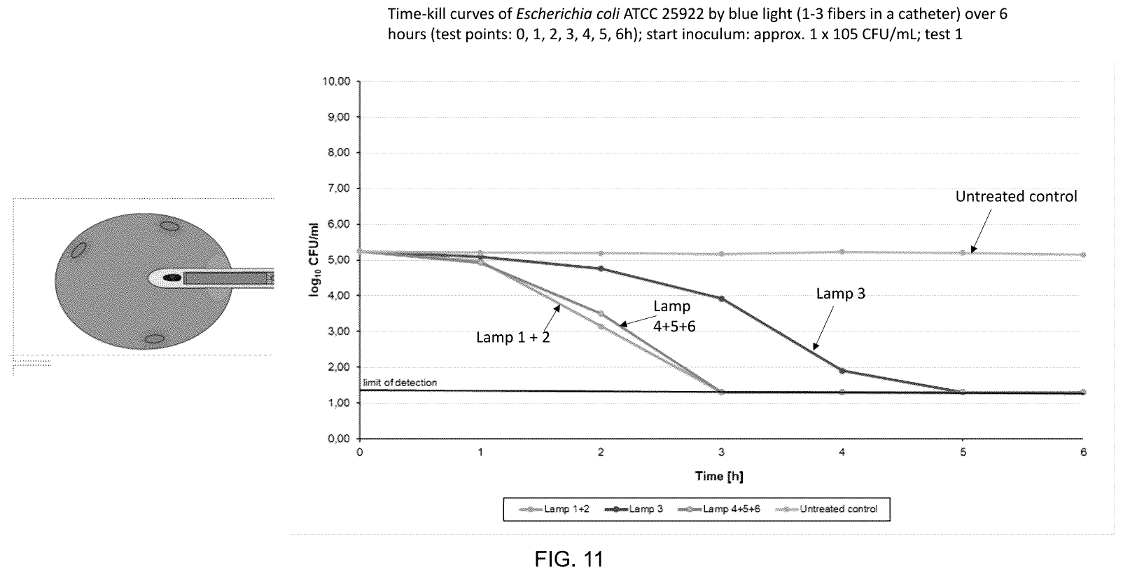

For example, FIG. 11 illustrates a graph showing exemplary time-kill curves of Escherichia coli by blue light, using 1-3 optical fibers, over a 6 hour period.

Similarly testing with the system described herein has shown that a single fiber is capable of achieving 3 log reduction either within the lumen, or on the outside of the catheter in an hour (for example, with a silicone catheter).

The use of the system having a catheter and optical fibers at the onset of Foley catheter delivery is a very simple and easy method towards providing a prophylactic treatment of CAUTI.

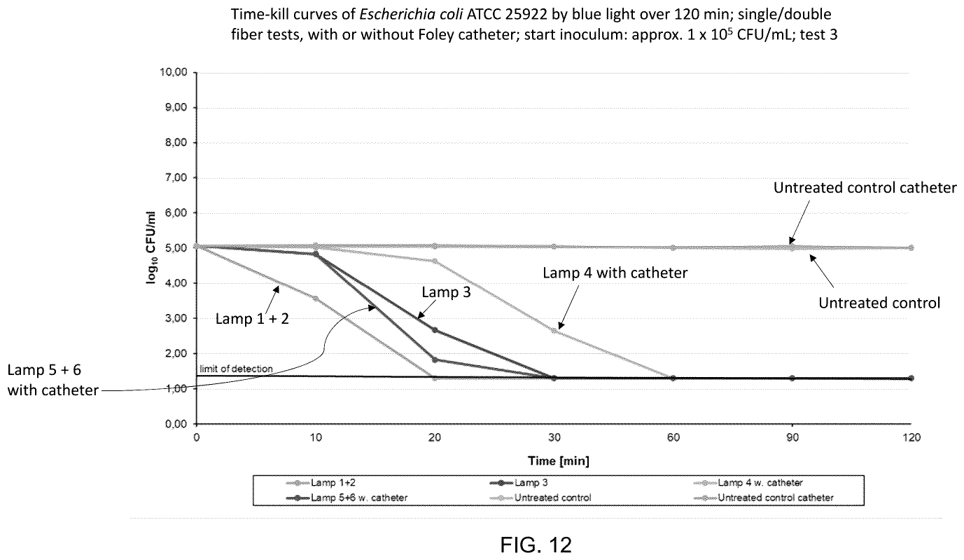

For example, FIG. 12 illustrates a graph showing exemplary time-kill curves of Escherichia coli by blue light over 120 minutes.

The controlled delivery of blue light in frequencies that can cause the death of the bacteria that causes infections can be achieved using one or more light fibers inserted through a catheter using the fitting described herein. The use of light in the formation and transfer of molecular oxygen on a cellular level, forming a reactive singlet oxygen, where this oxidizing species can destroy proteins, lipids, and nucleic acids causing cell death and tissue necrosis. The methods and systems provide secondary fluids, e.g., H₂O₂, that will enhance the death of the bacteria, wherein the blue light has weakened/damaged the outer shell of the bacteria, and the O₂ from the peroxide accomplishes the final oxidation destroying/causing the death of the bacteria.

Blue light has demonstrated antimicrobial properties against a range of microbes, including but not limited to gram-positive and gram-negative bacteria, mycobacteria, molds, yeasts, dermatophytes, and similar pathogens. Antimicrobial blue light having wavelengths between about 400 nm to about 470 nm can be used as alternative to antibiotics.

In some embodiments, the device, systems and methods disclosed provide for an application of light to kill an infection which creates the formation and transfers energy to molecular oxygen, thus forming the reactive singlet oxygen. This oxidizing species can destroy proteins, lipids, and nucleic acids causing cell death and tissue necrosis. The instant disclosure's application of light creates molecular oxygen, thus forming the reactive porphyrins. For example, during treatment, electromagnetic radiation having wavelengths in the visible spectrum (i.e., visible light above 395 nm, by non-limiting example) reacts with naturally produced and/or concentrated “endogenous” chromophores (porphyrins). At least one effect of the application of the electromagnetic radiation (illumination) is that the light in conjunction with or in combination with the porphyrins produces necrosis or cell death to the bacteria as evidenced by the microorganism's inability to divide. It is noted that the application of treatment of the instant disclosure provides treatment without the addition of ancillary drugs or chemicals, which can be considered as a “holistic” killing treatment or approach to fighting infection.

As a light-based disinfection approach, antimicrobial blue light (aBL), particularly in the wavelength range of 400 nm-500 nm, has an intrinsic antimicrobial effect. Compared to traditional photodynamic therapy, aBL therapy excites the endogenous chromophores of bacteria, and thus does not require the addition of exogenous photosensitizers. Furthermore, in comparison to ultraviolet irradiation, aBL shows much less detrimental effects in mammalian cells. The bactericidal activity of aBL is non-specific, and many microbial cells, including various antibiotics resistant strains, are highly sensitive to this treatment. aBL therapy has previously shown promise as a treatment for various clinical pathogens, including, but not limited to Pseudomonas aeruginosa, Acinetobacter baumannii, methicillin resistant Staphylococcus aureus (MRSA), and Candida albicans, and other pathogens.

In some embodiments, the light intensity will be uniform over the entire length of the one or more optical fibers used to deliver the blue light to the treatment site. This can be achieved, for example, by removing the cladding in specific configurations such that the intensity of light that is passed to the tissue through the areas of the fiber without cladding is uniform along the entire length thereof, as will be explained in more detail below. In some embodiments, this allows light to be emitted down the entire length of the fiber from the side of the fiber to treat any length of tissue.

If a prescribed dose (for example, intensity or some other measurement associated with the light) is defined as the means to achieve an antimicrobial effect, then that dose/amount of energy needs to be delivered over the entire length of the fiber for the affected area to be treated (except in the case of an infection being confirmed to a single location where illumination can be directed similar to the effect of a flashlight or spotlight).

In some embodiments, it is possible to define a specific energy that is required to remediate a specific bacteria. This can include the energy emitted by the fiber, and a time of the exposure. This can be used to define the joules required to kill a bacteria. As the treatment often involves the treatment of elongate areas, the illumination of the fiber should be even over the length of the fibers, and also even in a circumferential manner. Thus, in some embodiments, the light is not only delivered evenly in a single angle off of the fiber but is also delivered evenly or equally in a circumferential manner.

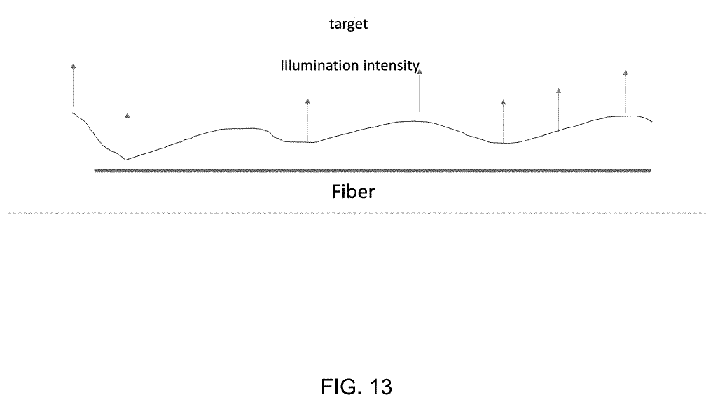

The light delivered by the fiber needs to be even over the length of the fiber such that the correct power over the length of the fiber can be provided, at even powers at short lengths over the fiber to achieve the antimicrobial effect, as shown in FIG. 13. If there is an imbalance in the power over the length of the fiber, there can be high spots and/or low spots, and the patient suffers as there isn't any way to achieve a balance in treatment. If there are high spots and/or low spots (areas of increased intensity and/or areas of decreased intensity), then there is not even power deposition to the targeted area needing treatment. This would correlate to overmedication or undermedication of the treatment site. Thus, the correct power can be selected for an even power distribution over the length of the fiber.

In some embodiments, uniform light delivery along the length of a fiber can be achieved by virtue of a variable helical spiral. Removal of cladding in this manner allows the system to maintain the same energy deposition over the length of the fiber such that the top and the bottom are even and light emanating from all planes of the fiber are uniform. This allows for 360 degrees of light over the length of a fiber.

The type of light fiber can vary, and can include traditional fiber optics, telecommunication fiber, or plastic fiber optics that can be efficient in the transmission of light with minimal light loss. It should be noted that with any form of diffusing/diffusion light fiber, the intensity of the light will decrease over length of the fiber dependent upon the amount of light being diffused (length and/or area).

A process of even diffusion of the light in the cladding over the length of the fiber results in stronger intensity at the initiation end of the fiber and an ever decreasing amount as distance is increased from the initiation source. This reduction in optical power and intensity negates it's use in the curing of photodynamic implants, as the intensity at the distal end has weakened significantly (or the increased power to achieve curing at the distal end of the fiber has been increased so significantly that there is an overpowering of the fiber at the proximal end). Thus, a variable helix of a cut in the cladding, spiraling down the fiber, with the spiral getting tighter and tighter as the light is bleeding out allows for even light dispersion over the length of the fiber.

In some embodiments, an antimicrobial system can include an optical fiber having a diameter in the range of 1 mm to 20 mm, with a light emitting helical coil on the circumference of the fiber. The illumination of the fiber is delivered radially from the fiber outwards. Illumination frequencies are in the visible spectrum from about 400 nm to about 475 nm.

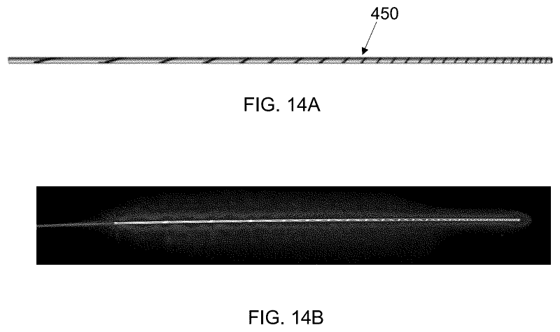

Thus, in some embodiments, a fiber 450 can have a spiral/helical coil of cladding that can be removed, allowing light to escape from within the fiber to affect the ABLP process, as shown in FIG. 14A and FIG. 14B. The cladding is removed such that the light intensity along the length of the fiber is uniform. Traditionally, PMMA (acrylic) comprises the core (96% of the cross section in a fiber 1 mm in diameter), and fluorinated polymers are the cladding material. Since the late 1990s much higher performance graded-index (GI-POF) fiber based on amorphous fluoropolymer (poly(perfluoro-butenylvinyl ether), CYTOP) has begun to appear in the marketplace. Polymer optical fibers are typically manufactured using extrusion, in contrast to the method of pulling used for glass fibers.

In some embodiments, cladding is removed physically (i.e., scratching the surface in a very precise method using, for example, a diamond tipped cutting head, a razor, or scalpel blade) which can reveal the fiber and allow light to emanate through the space in the cladding In some embodiments, cladding is removed chemically from a polymer optical fiber using organic solvents which can also be used to create etched portions of the fiber allowing the attenuation of light. Cladding can also be removed using low energy lasers to finely ablate the surface, water jet cutting, or with compressive dies to penetrate/break the surface of the cladding.

To provide for uniform light delivery, when the cladding is removed along the length of the fiber, the spiral can tighten as it progresses from a proximal end of the fiber to a distal end of the fiber. In some embodiments, the depth of the cut into the fiber can also increase from the proximal end of the fiber to the distal end of the fiber.

The greater penetration depth of the fiber towards the dispersion of light requires increased penetration depth as the light intensity also decreases through the attenuation or loss of light though the removed cladding area.

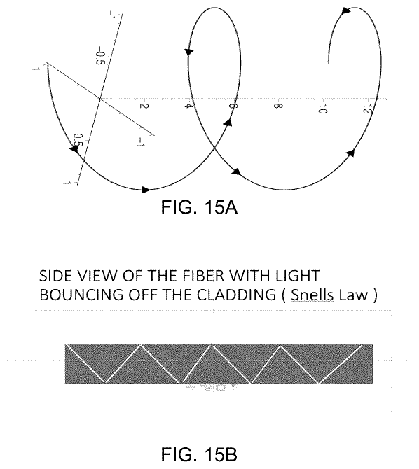

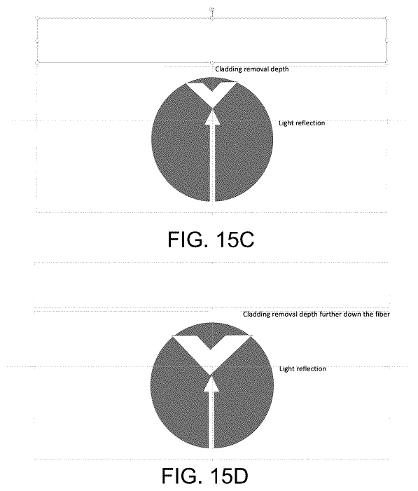

For example, a spiral design, as shown in FIG. 15A, FIG. 15B, FIG. 15C, and FIG. 15D, can be used that wraps around the fiber on the line of the spiral, where the cladding has been removed to allow light to escape. As the light escapes, the intensity of the light being emitted is reduced from 100% of the light at a proximal end of the fiber closest to a light source, and the light moves distally further and further down the spiral, there is less light coming out of the fiber (the light intensity decreases). In order to resolve the issue, the pitch can be increased to narrow the gap between the two spirals and to increase the amount of light hitting the target. However, as the light moves further and further down the fiber and the intensity is dropping, the depth of the cut in the cladding can be increased to decrease the distance that the light within the fiber needs to transit before exiting the fiber. Thus, in order to achieve the necessary amount of light along the entire fiber length, there is a balance of the spiral and the depth of the cladding cuts to achieve an even light distribution over the length of the fiber. For example, an initial cut in the fiber can be shallow such that only the cladding is removed. The cuts can become deeper distally along the length of the fiber (i.e., thousands of an inch deeper).

In some embodiments, the cladding is removed from only certain portions of the fiber. In some embodiments, the cladding is removed 360 degrees around the fiber. In some embodiments, the cladding is only removed in a 180 degree orientation, as shown in FIG. 17, with the cut side outwards. As shown in FIG. 17, the uncut side of the fiber 450 can be positioned against a balloon 452. The 180 degree cut side is exposed to the endosteal surface. By having the spiral cut only on 180 degrees of the fiber, the intensity of the light increases. The reduction in the cut cladding can increases the output efficiency. In some embodiments, the cladding is removed in a 90 (45+/−) degree orientation, with the 90 degree cut side exposed to the endosteal surface. This reduction in the cut cladding can also increases the output efficiency. In some embodiments, the cladding is removed in a 120 (60+/−) degree orientation, with the 120 degree cut side exposed to the endosteal surface. The reduction in the cut cladding can increase the output efficiency. It will be understood that any amount of the circumference of the cladding can be removed from the fiber to control the amount of light from the fiber and the area of tissue being exposed thereto. The cladding can be removed in the direction of intended light delivery.

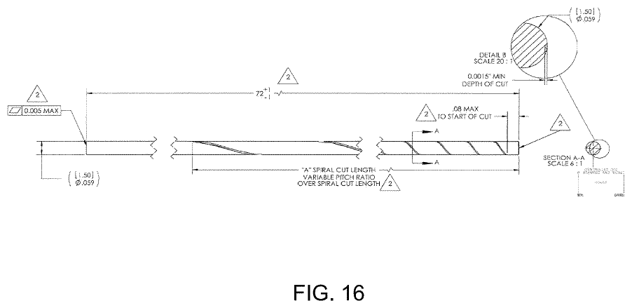

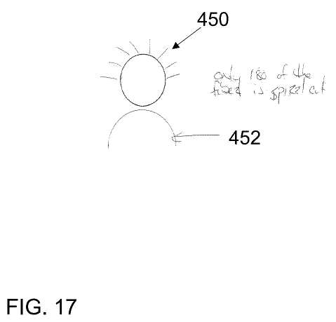

Based on cladding removal, as shown in FIG. 16, energy dissipation remains the same or substantially the same as length increases. Cladding cut width is same, but the depth of cut increases over the length so light emanates stronger toward the distal end. A helical spiral can be cut with a slower pitch at proximal end, and the pitch can increase as it moves toward the distal end, creating a tighter spiral.

In some embodiments, rather than removing a portion of the cladding from the fiber to achieve uniform light energy delivery/power deposition along the length of the fiber, one or more fibers can be used to deliver the light energy uniformly does not include cladding. When using a fiber without cladding, the fiber can be overcoated with a light diffusing membrane. The over coating can be applied (or an extrusion) where the leaking of light through the diffusion membrane is low at the proximal end of the fiber (or light intensity side) and the diffusion can gradually increase as the fiber gets longer towards the distal end of the fiber. The outer membrane can be scaled to allow for uniform/even light and power deposition along the length of the fiber.

A diffusive membrane can be deposited in specific thicknesses or patterns on the fiber to achieve uniform delivery of light. In some embodiments, segments of diffusive material can be applied to the outer surface of the fiber in an arrangement that provide uniform light along the length thereof. In some embodiments, a diffusive coating (e.g., a spray coat, dip coat, or ionic deposition coating) can be applied to the fiber such that the thickness of the coating decreases along the length from the proximal end to the distal end of the fiber. The thinning of the coating towards the distal end allows for an increase in the amount of light diffusion through the coating from the proximal to the distal end such that the end result is uniform delivery of light along the length of the fiber. As described herein throughout the disclosure and the various embodiments, the energy across the length of the fibers (the linear deposition of even power) is uniform such that the bacteria or other microbe at the treatment site will be killed at an even rate.

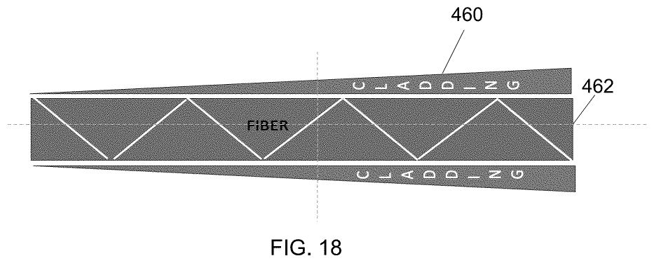

In some embodiments, the modification to the coating is that it can be thicker or less transparent to minimize light transmission through the fiber (and decrease loss) at the proximal end of the fiber, as shown in FIG. 18. The coating or cladding 460 can decrease in thickness from the proximal end to the distal end of the fiber 462, increasing the transmission at the end of the fiber. For example, it can be assumed that the amount of light transmission through the cladding is sufficient at the proximal end and can match to the amount of light transmission at the distal end.

### EXAMPLES

Various studies applying blue light to a sample were performed. For example, one fiber and two fibers experiments were performed with 5 LED lamps 1, 2 and 5 LED lamps HP 3, 4, 5, 6.

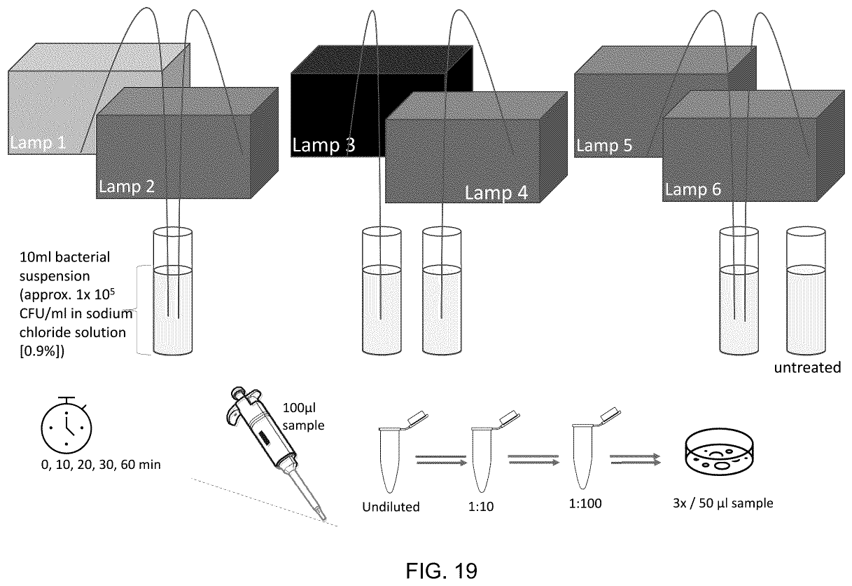

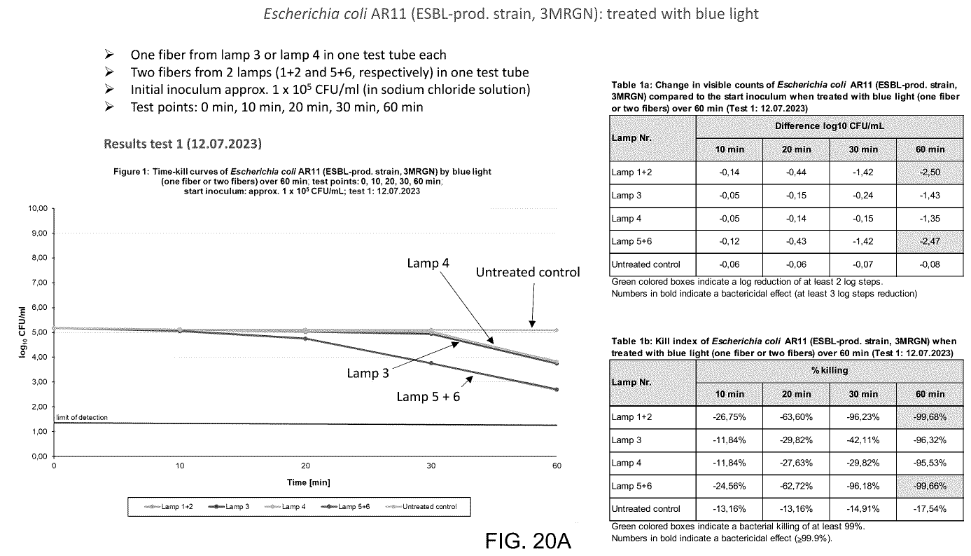

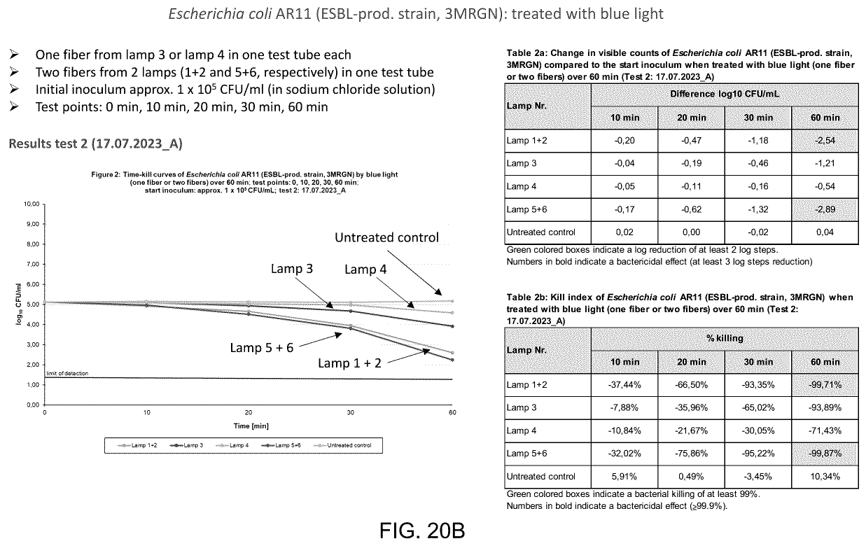

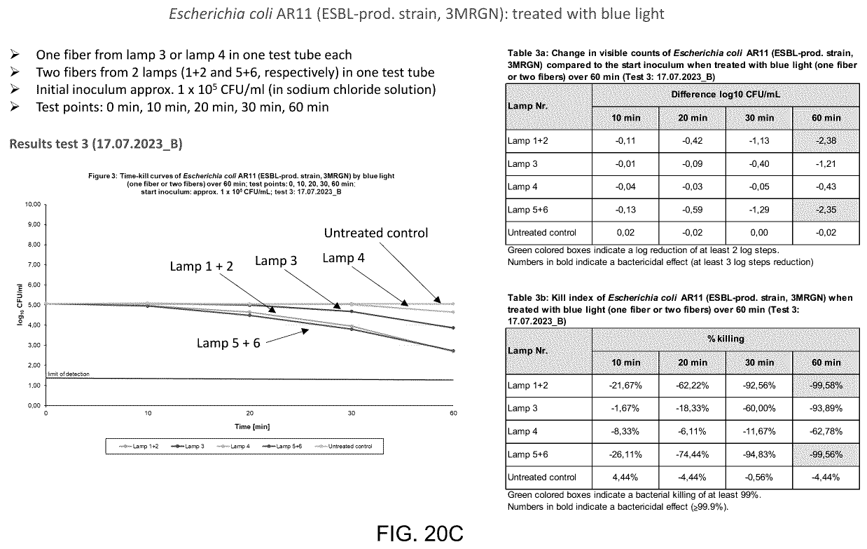

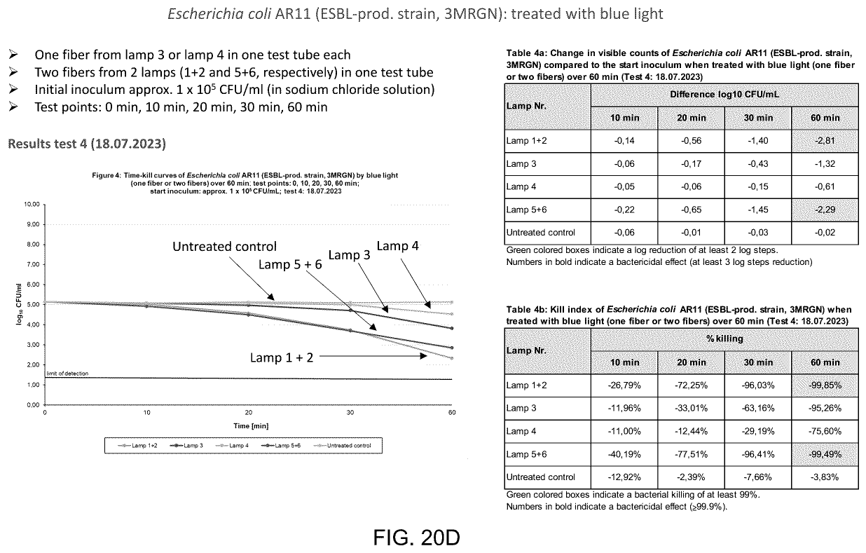

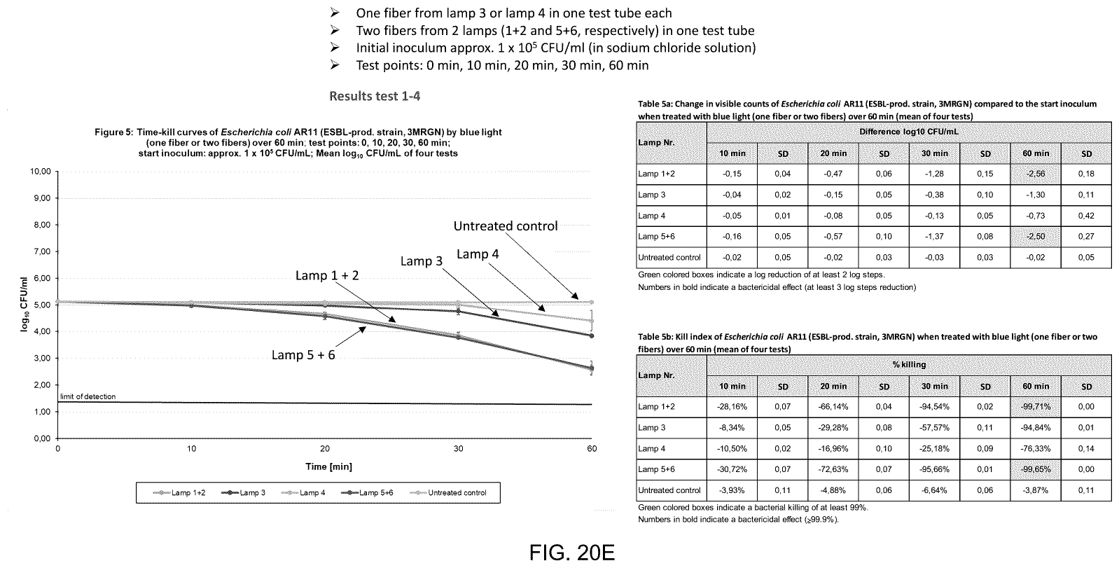

The test set up and results shown in FIG. 19 and FIGS. 20A, 20B, 20C, 20D, and 20E are for a test strain of Escherichia coli AR11 (ESBL-prod. strain, 3MRGN). Four experiments were run for 10 minutes, 20 minutes, 30 minutes and 60 minutes and data was recorded related to the amount of the test strain that remained in the sample. The graphs shown in FIGS. 20A-20D illustrate the amount of the test strain in the sample versus time of exposure to blue light. The graph and data in FIG. 20E illustrates the overall findings of an aggregate of the experimental data shown in FIGS. 20A-20D, showing a reduction in the test strain due to blue light exposure. For example, there is a 3 log reduction in the test strain at 60 minute blue light exposure.

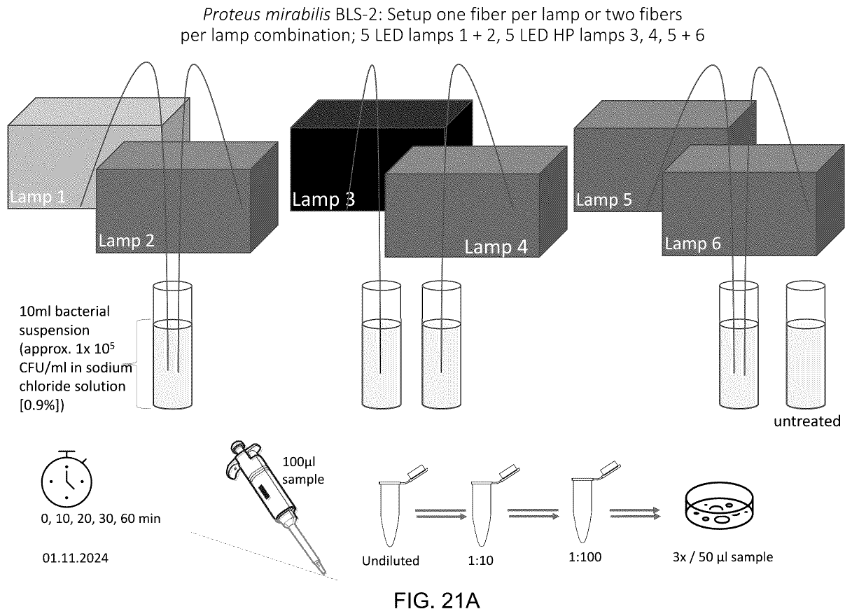

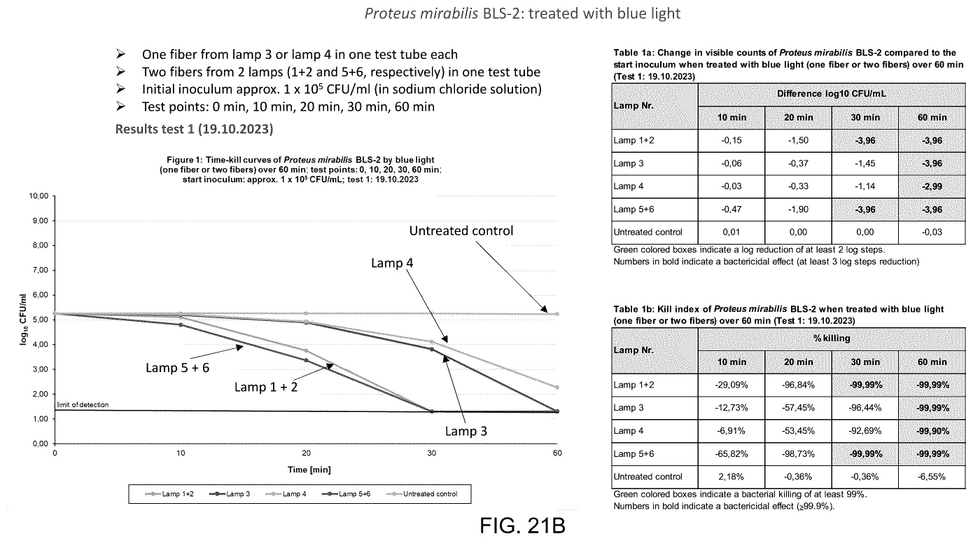

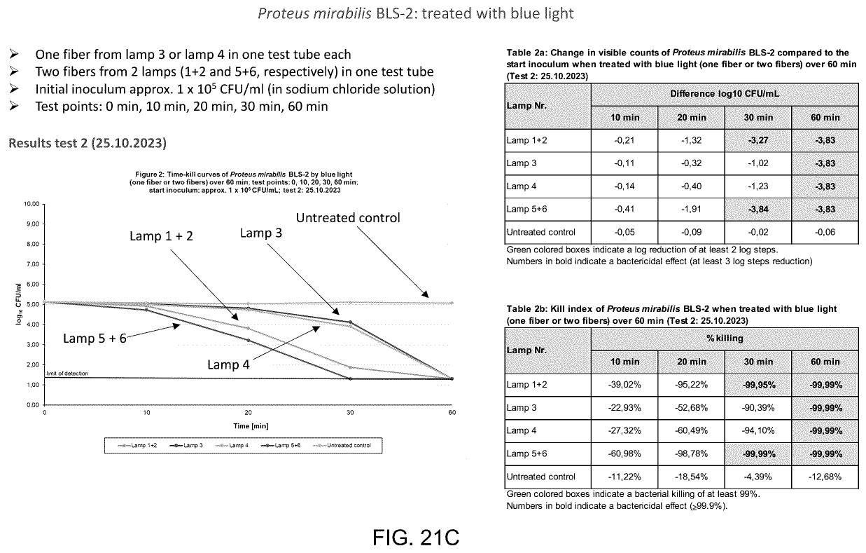

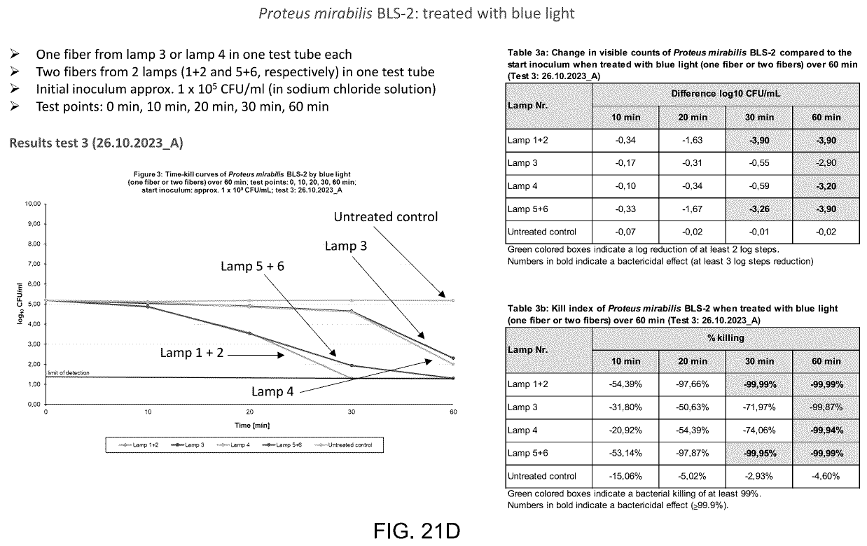

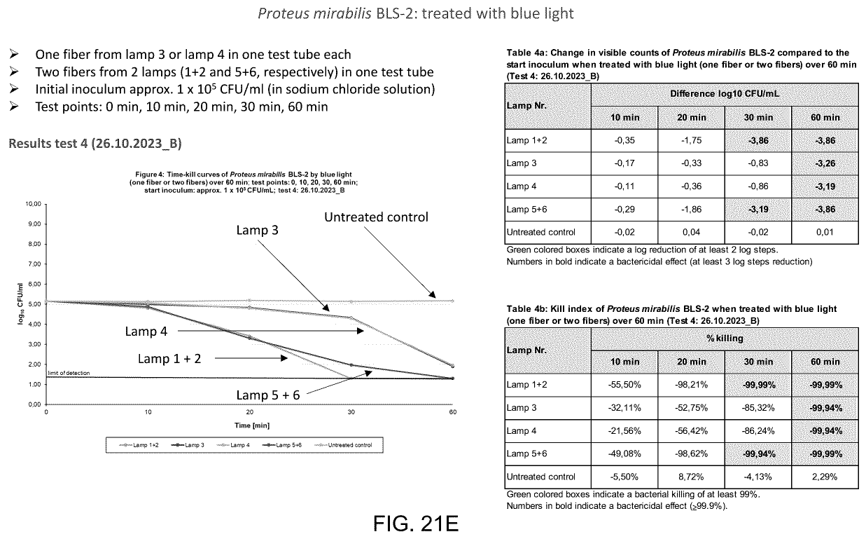

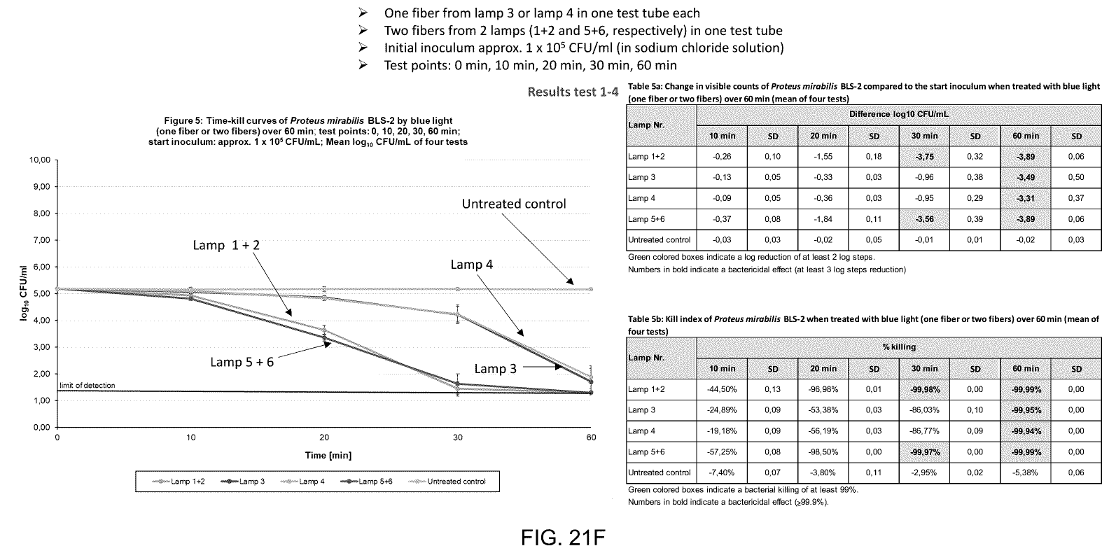

The test set up and results shown in FIGS. 21A-21F are for a test strain of Proteus mirabilis BLS-2. Four experiments were run for 10 minutes, 20 minutes, 30 minutes and 60 minutes and data was recorded related to the amount of the test strain that remained in the sample. The graphs shown in FIGS. 21B-21E illustrate the amount of the test strain in the sample versus time of exposure to blue light. The graph and data in FIG. 21F illustrates the overall findings of an aggregate of the experimental data shown in FIGS. 21B-21E, showing a reduction in the test strain due to blue light exposure. For example, there is a 3 log reduction in the test strain at 60 minute blue light exposure.

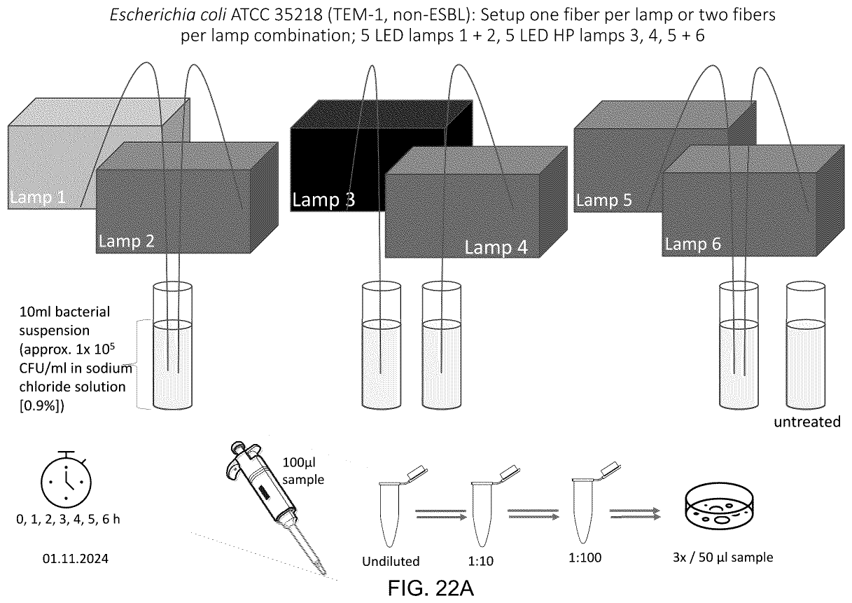

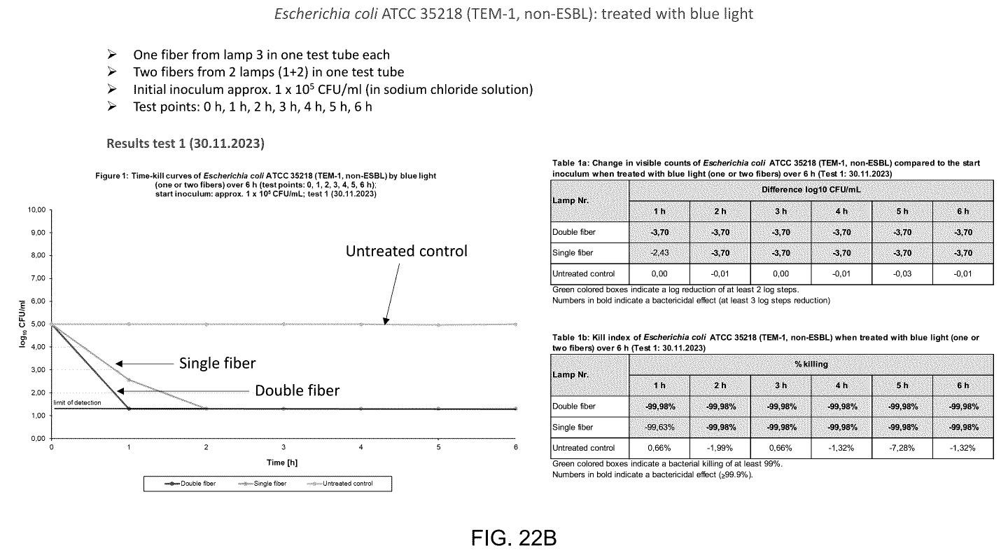

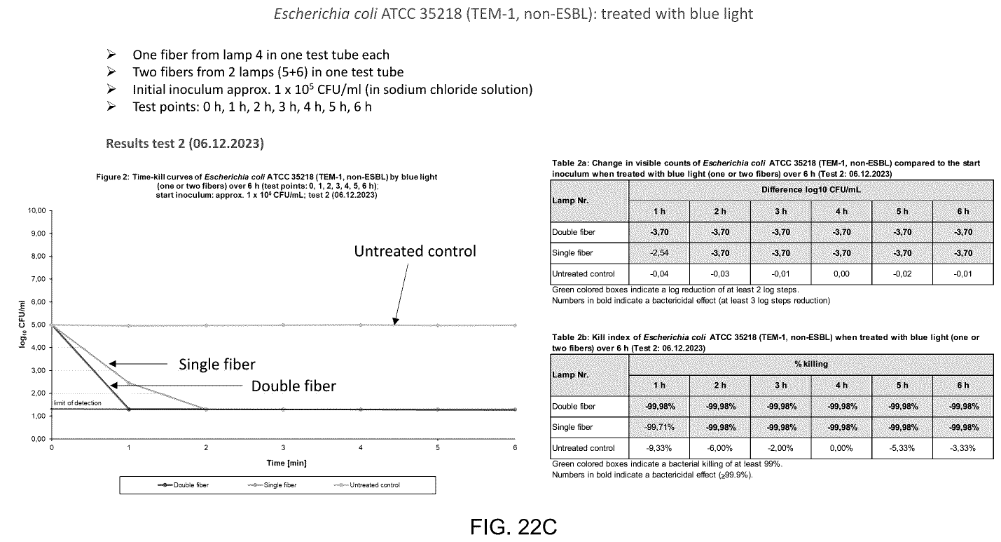

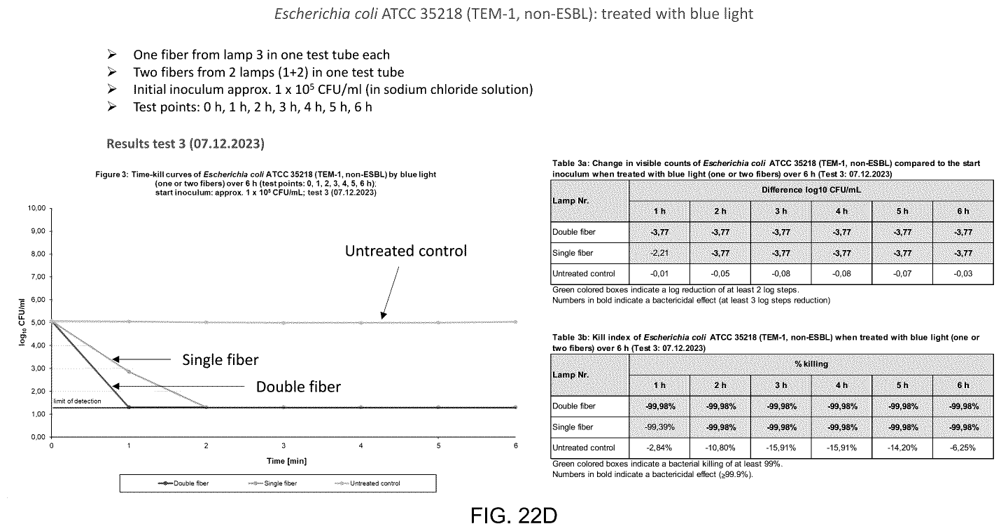

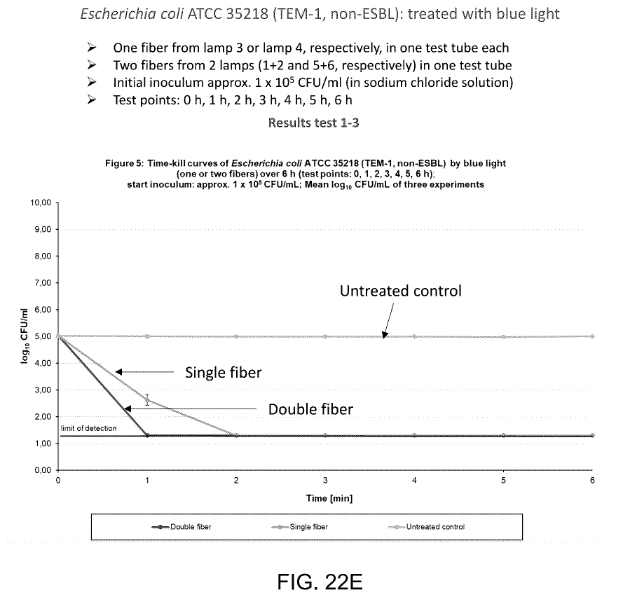

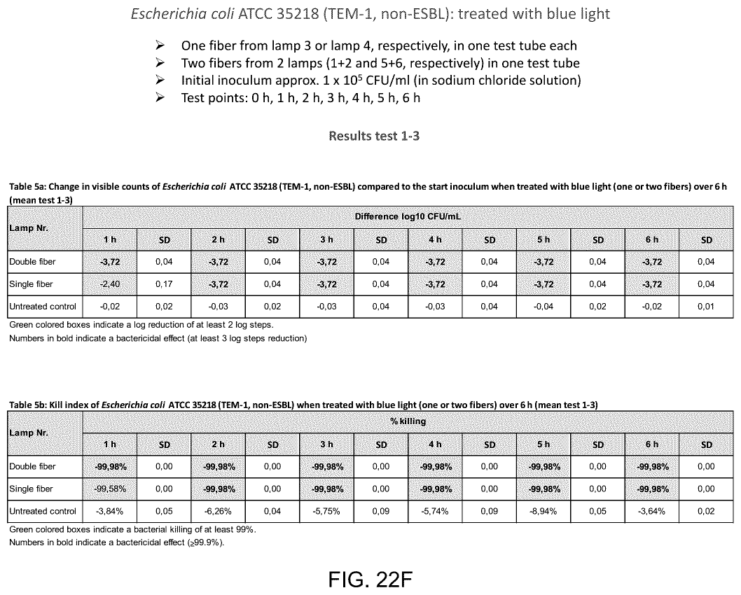

The test set up and results shown in FIGS. 22A-22F are for a test strain of Escherichia coli ATCC 35218 (TEM-1, non-ESBL). Three experiments were run for 1 hour, 2 hours, 3 hours, 4 hours, 5 hours, and 6 hours and data was recorded related to the amount of the test strain that remained in the sample. The graphs shown in FIGS. 22B-22D illustrate the amount of the test strain in the sample versus time of exposure to blue light. The graph and data in FIGS. 22E-22F illustrate the overall findings of an aggregate of the experimental data shown in FIGS. 22B-22D, showing a reduction in the test strain due to blue light exposure.

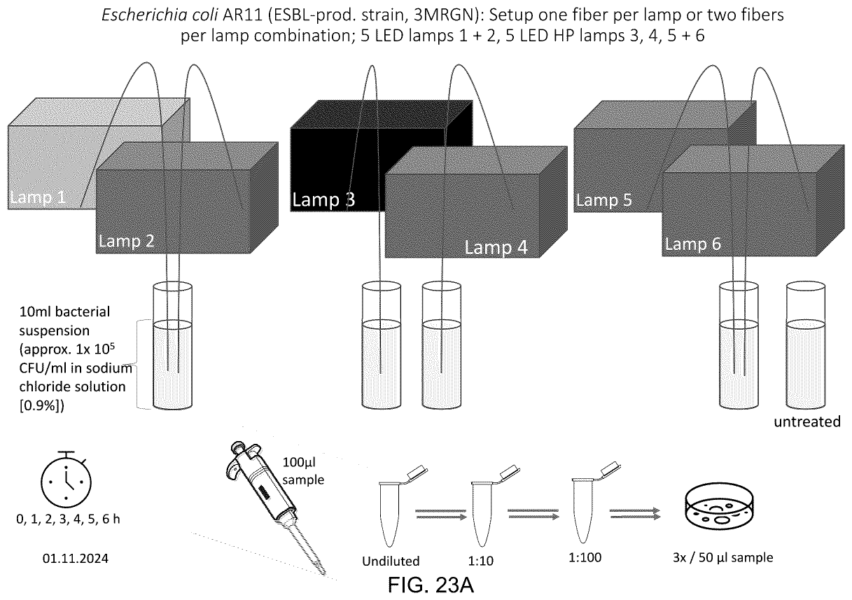

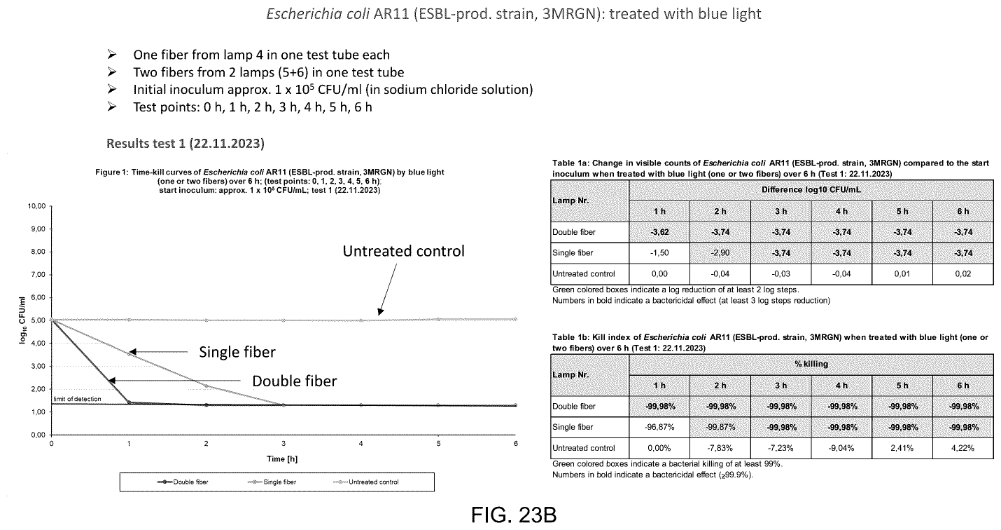

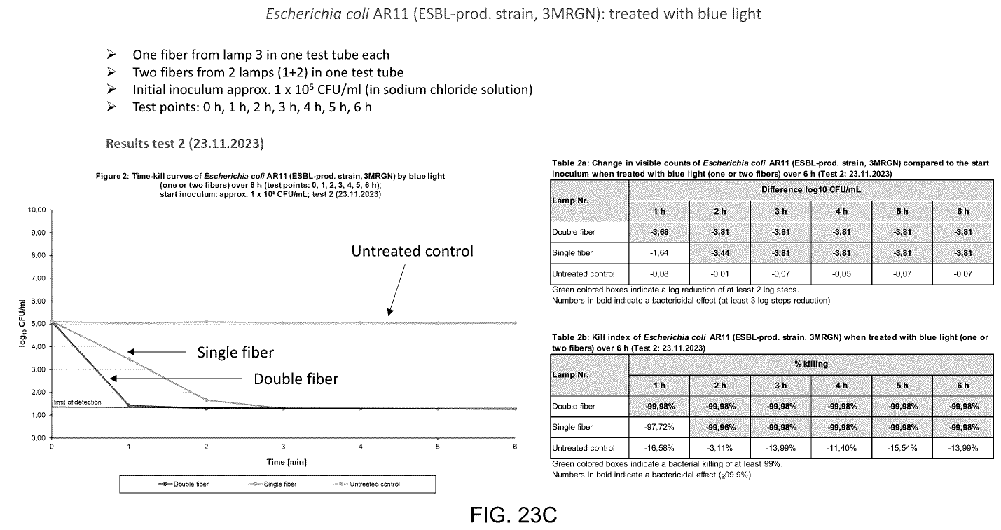

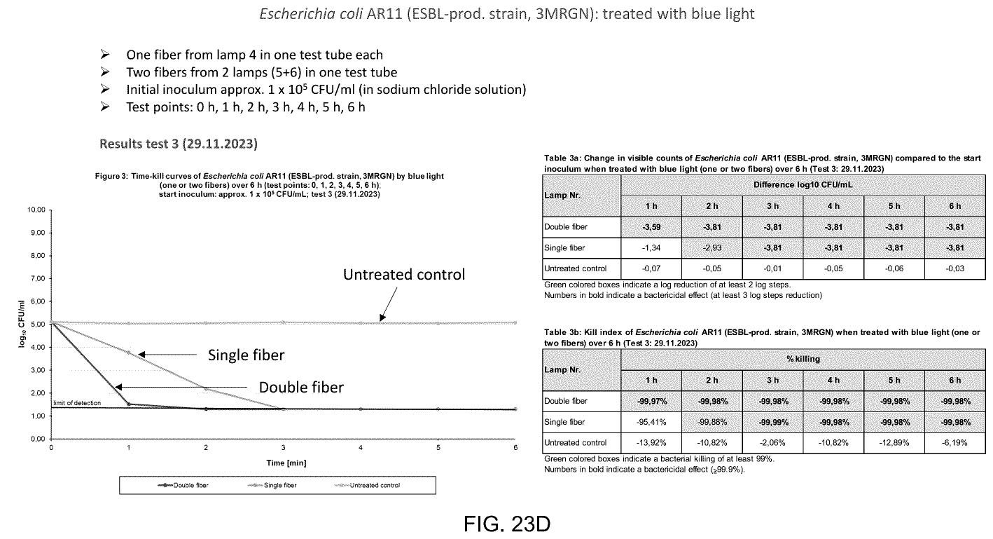

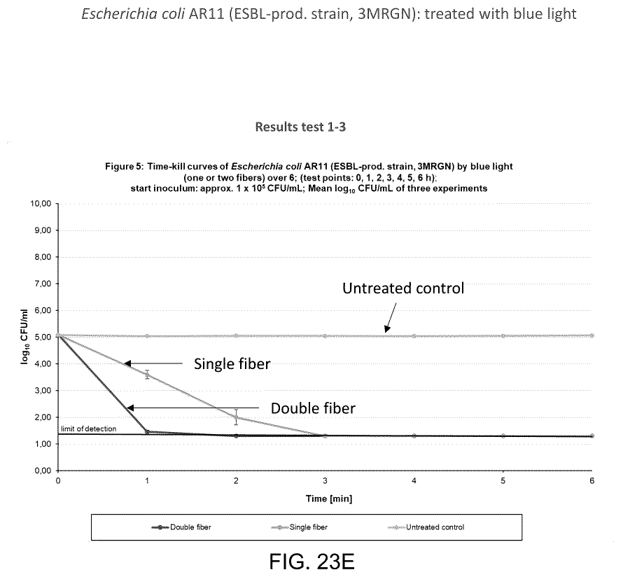

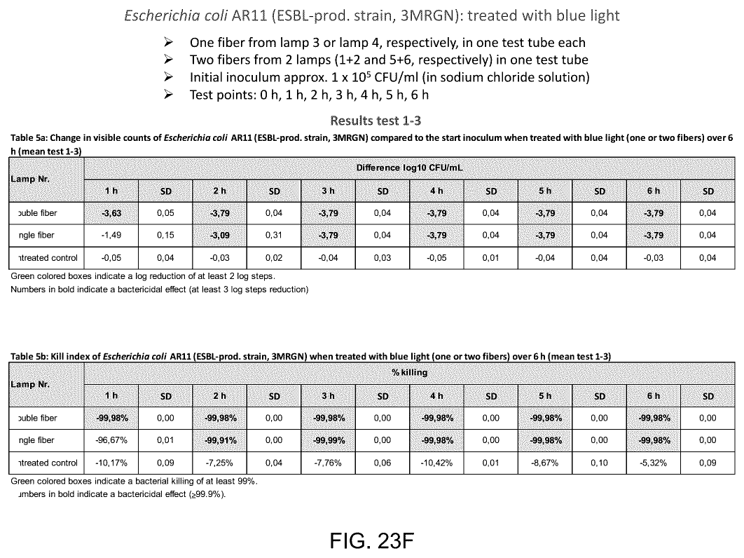

The test set up and results shown in FIGS. 23A-23F are for a test strain of Escherichia coli AR11 (ESBL-prod. strain, 3MRGN). Three experiments were run for 1 hour, 2 hours, 3 hours, 4 hours, 5 hours, and 6 hours and data was recorded related to the amount of the test strain that remained in the sample. The graphs shown in FIGS. 23B-23D illustrate the amount of the test strain in the sample versus time of exposure to blue light. The graph and data in FIGS. 23E-23F illustrate the overall findings of an aggregate of the experimental data shown in FIGS. 23B-23D, showing a reduction in the test strain due to blue light exposure.

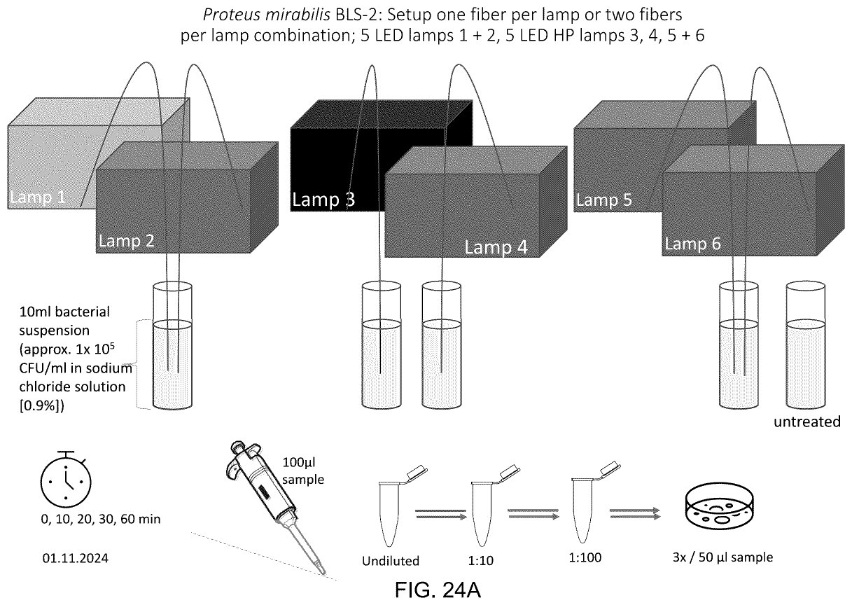

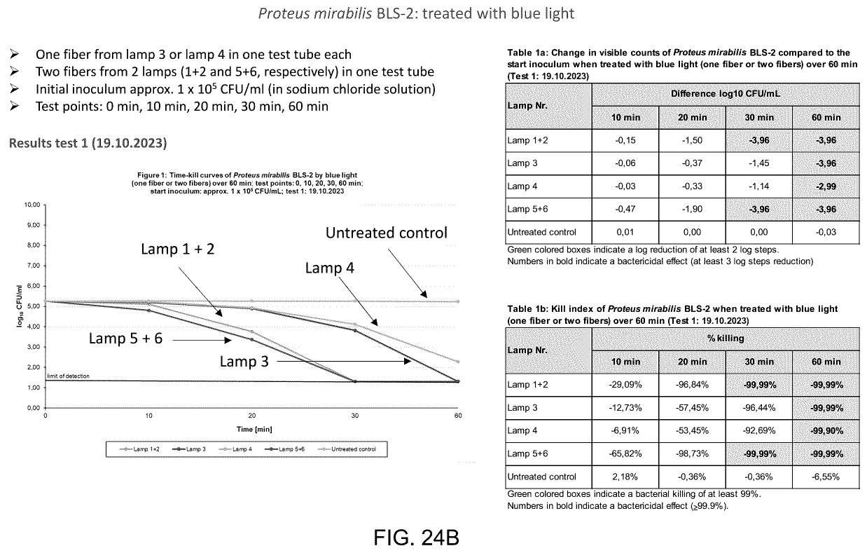

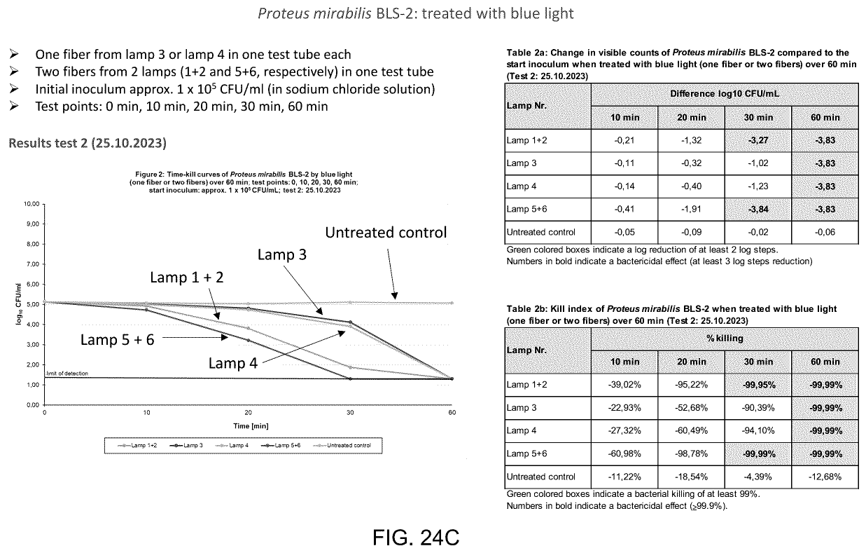

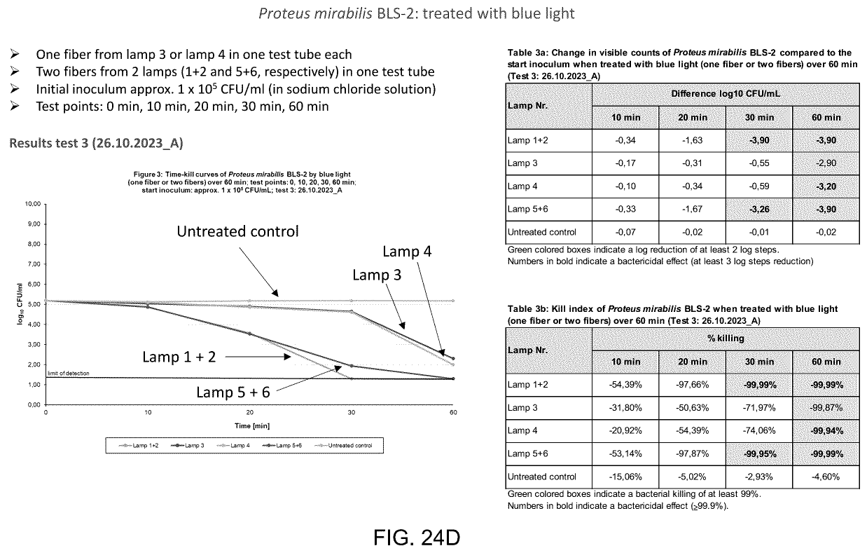

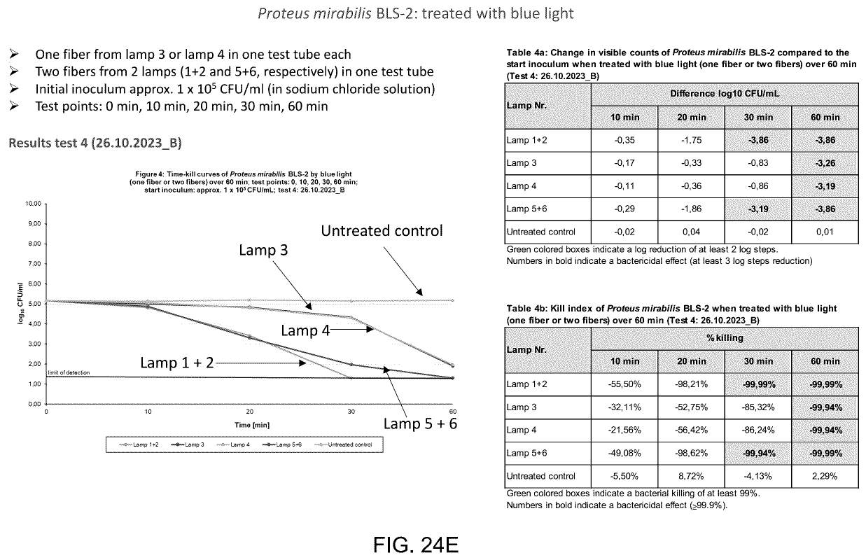

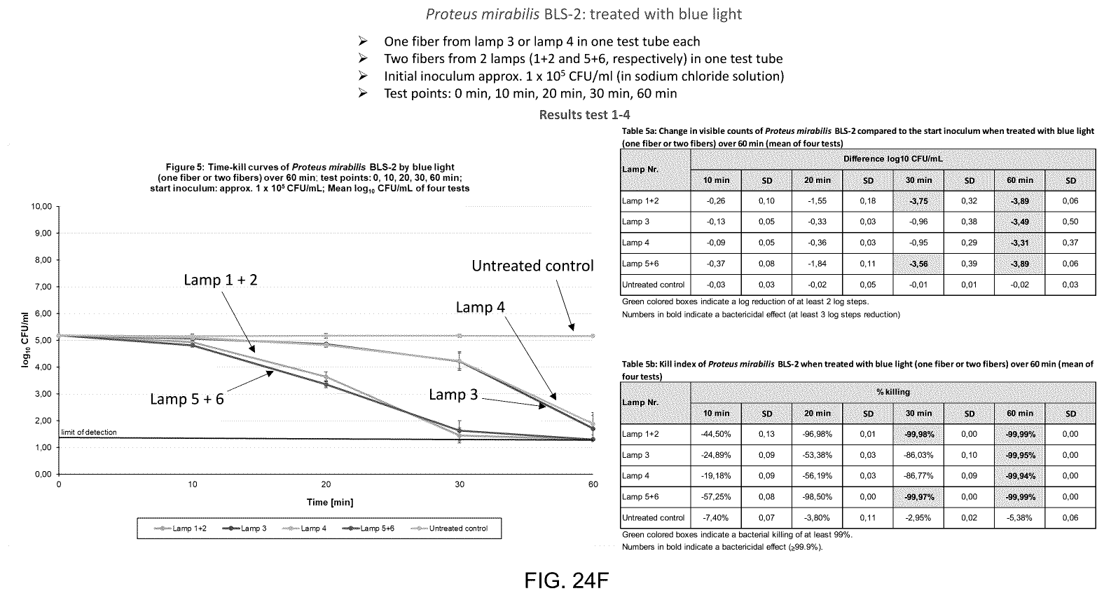

The test set up and results shown in FIGS. 24A-24F are for a test strain of Proteus mirabilis BLS-2. Four experiments were run for 10 minutes, 20 minutes, 30 minutes and 60 minutes and data was recorded related to the amount of the test strain that remained in the sample. The graphs shown in FIGS. 24B-24E illustrate the amount of the test strain in the sample versus time of exposure to blue light. The graph and data in FIG. 24F illustrates the overall findings of an aggregate of the experimental data shown in FIGS. 24B-24E, showing a reduction in the test strain due to blue light exposure. For example, there is a 3 log reduction in the test strain at 60 minute blue light exposure.

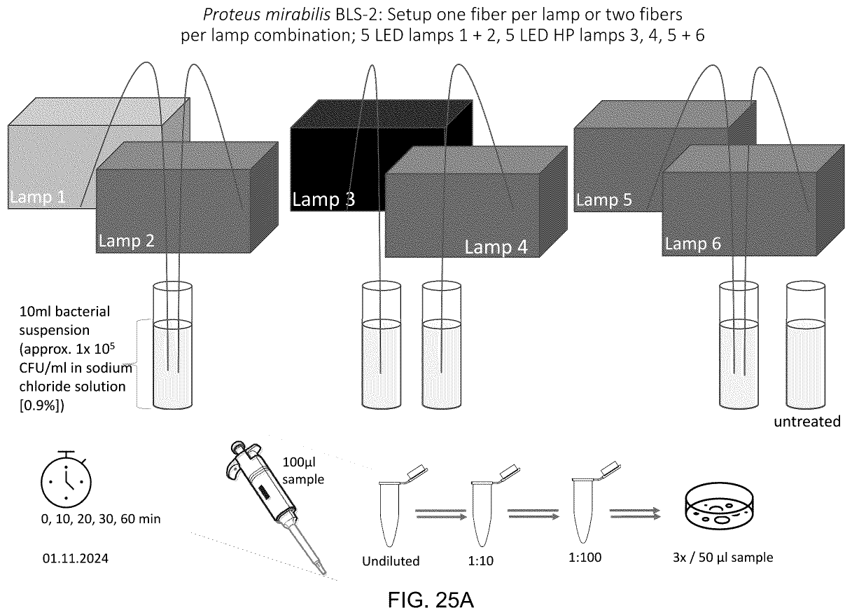

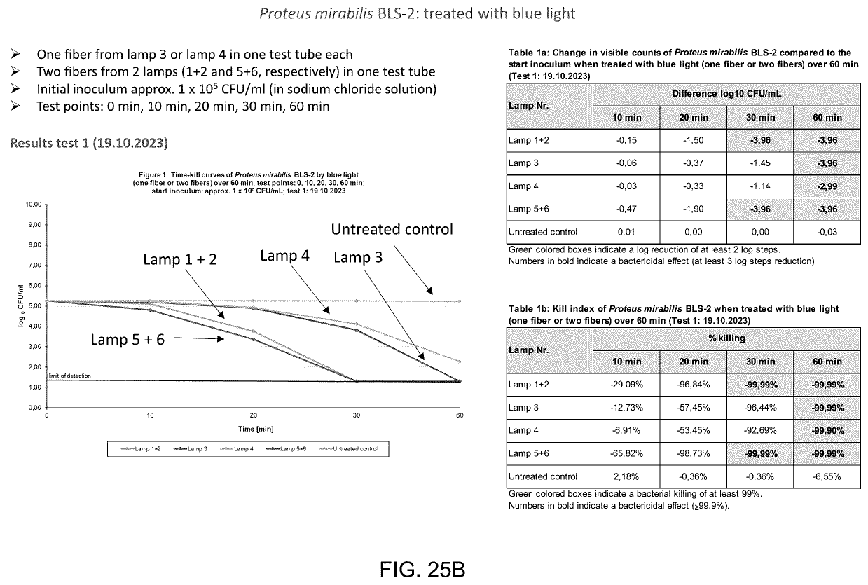

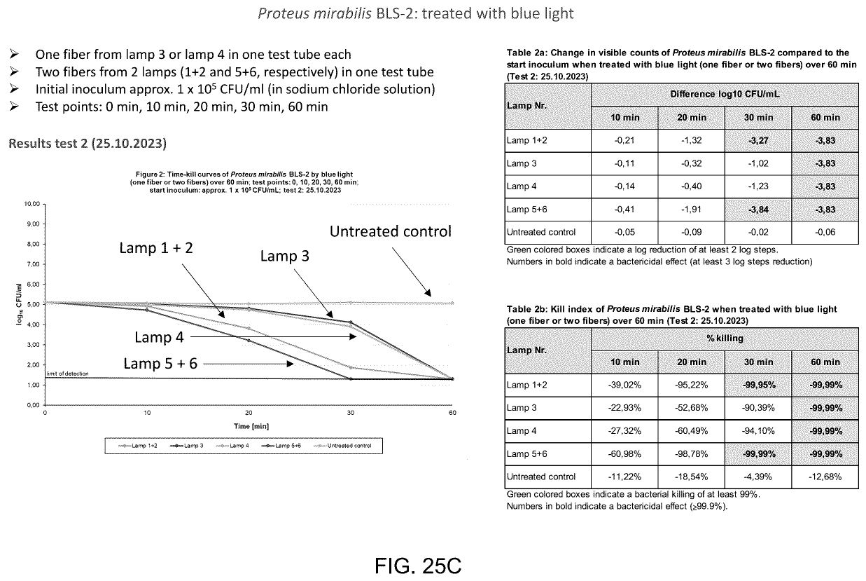

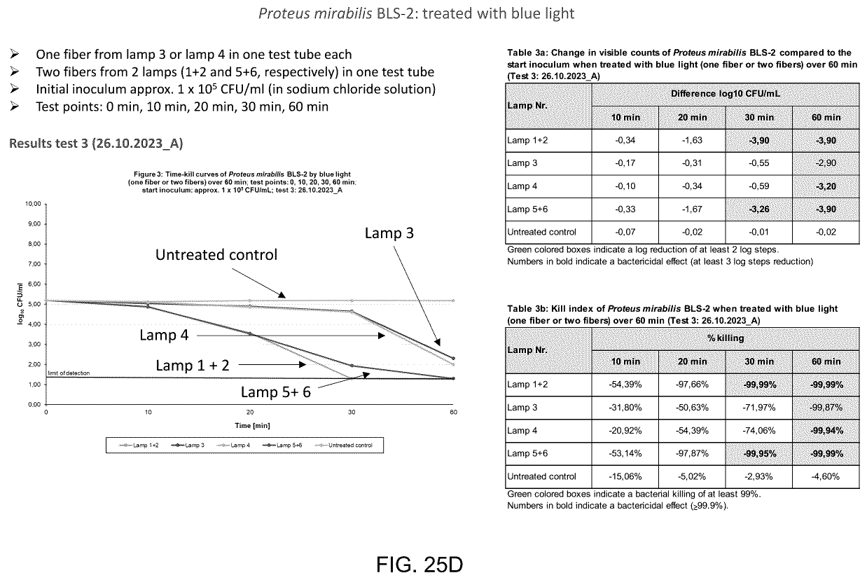

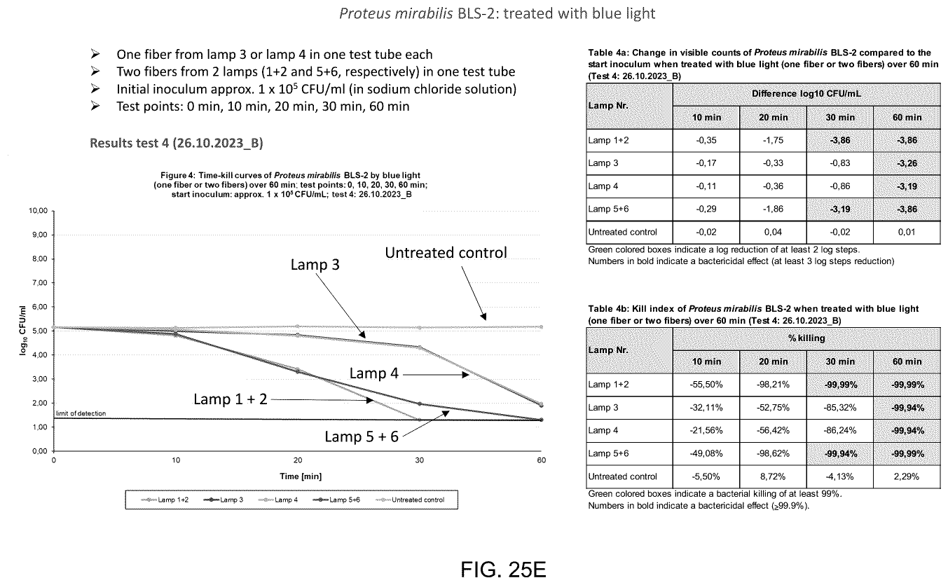

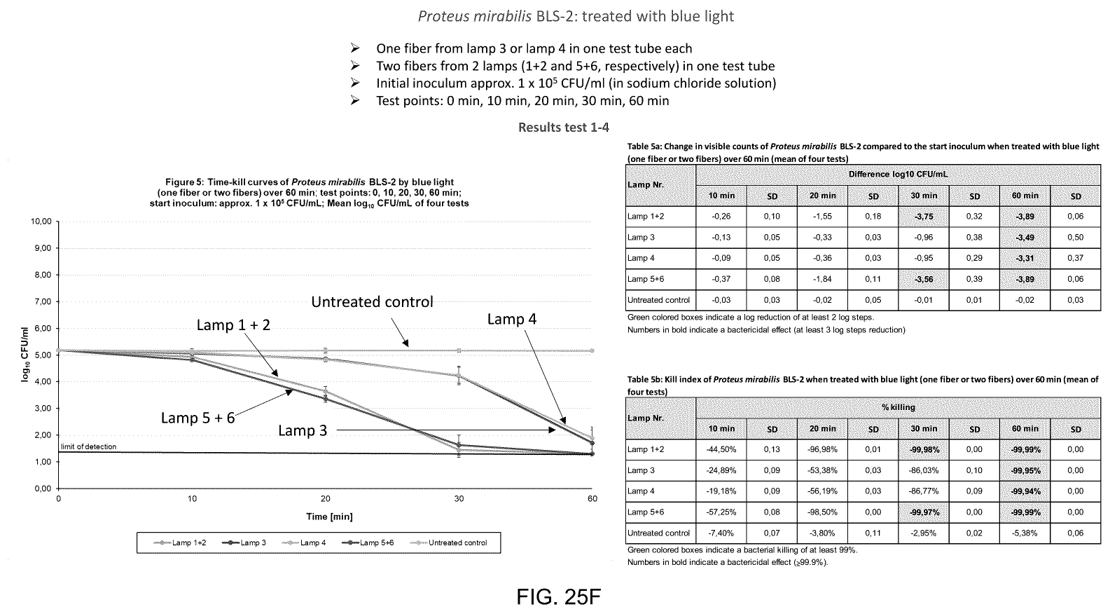

The test set up and results shown in FIGS. 25A-25F are for a test strain of Proteus mirabilis BLS-2. Four experiments were run for 10 minutes, 20 minutes, 30 minutes and 60 minutes and data was recorded related to the amount of the test strain that remained in the sample. The graphs shown in FIGS. 25B-25E illustrate the amount of the test strain in the sample versus time of exposure to blue light. The graph and data in FIG. 25F illustrates the overall findings of an aggregate of the experimental data shown in FIGS. 25B-25E, showing a reduction in the test strain due to blue light exposure. For example, there is a 3 log reduction in the test strain at 60 minute blue light exposure.

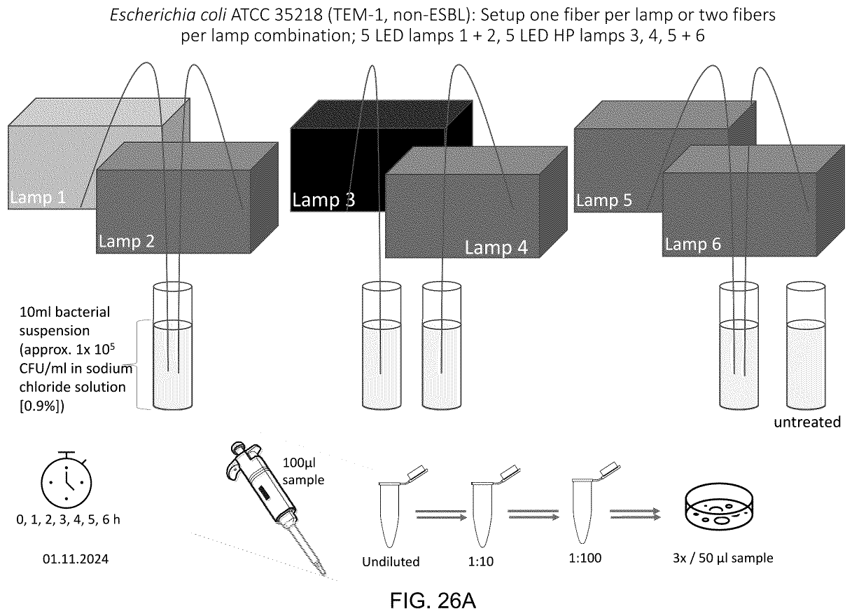

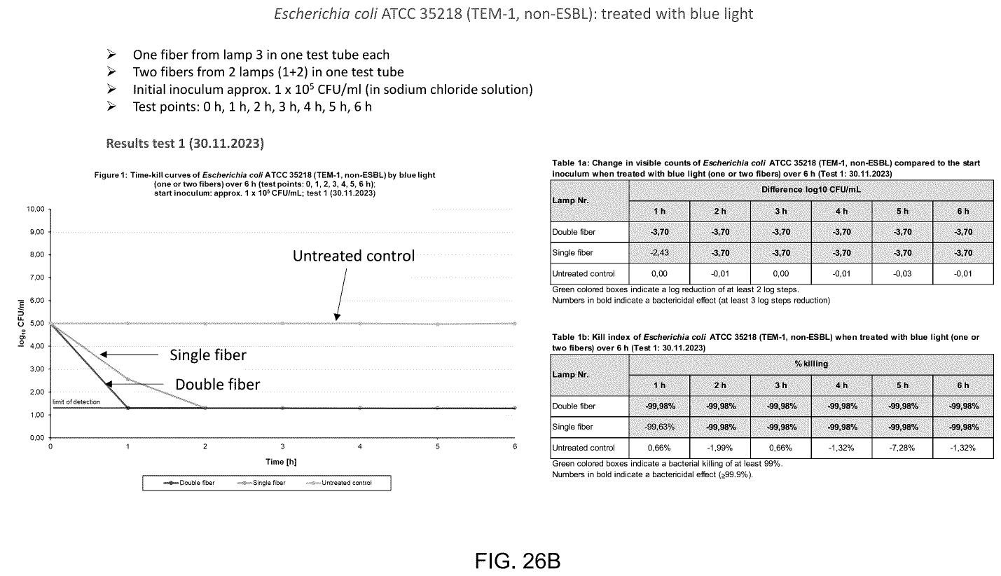

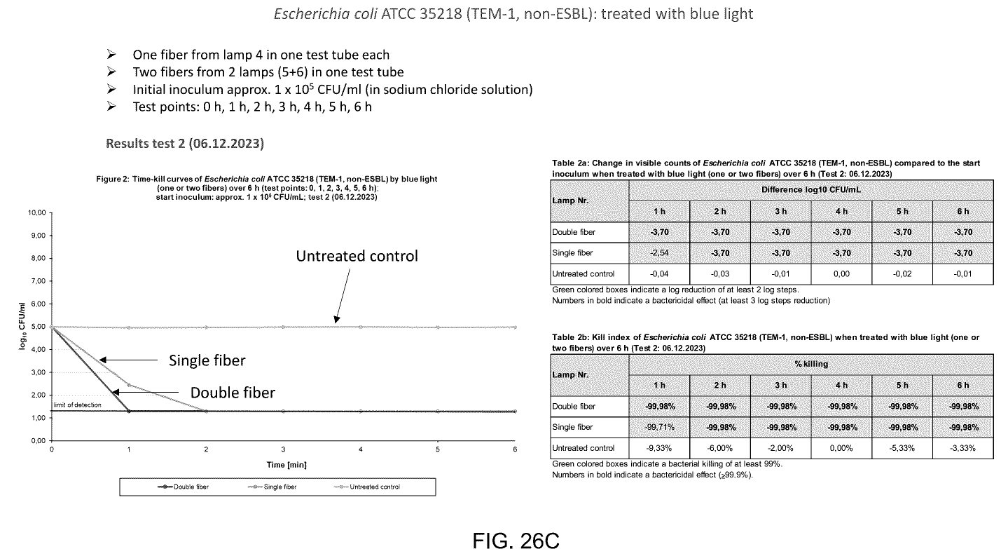

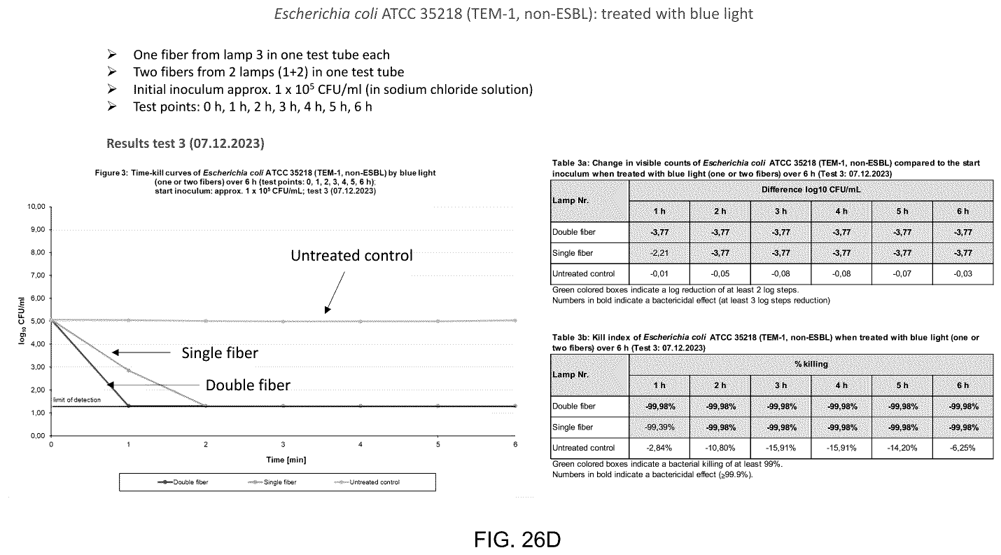

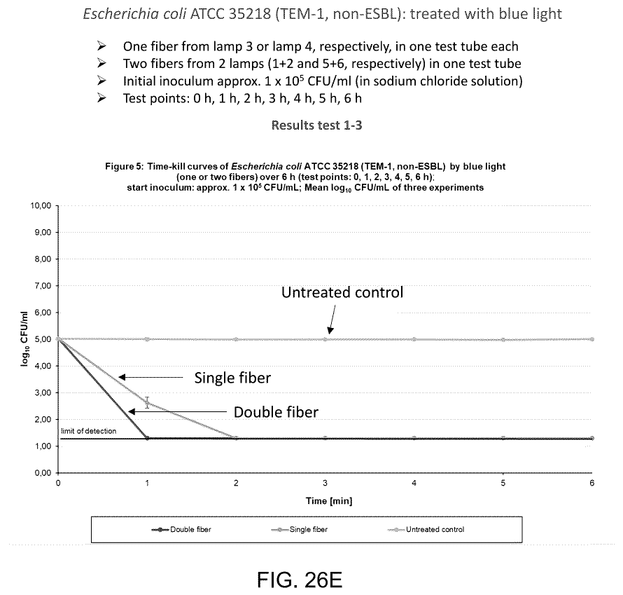

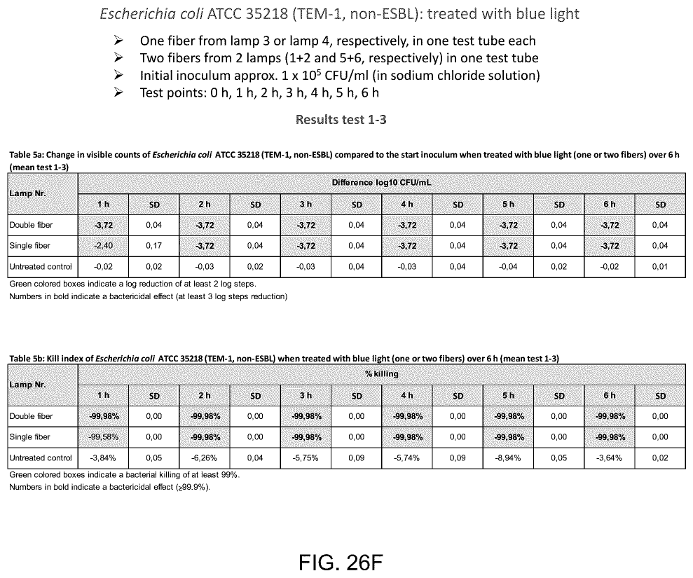

The test set up and results shown in FIGS. 26A-26F are for a test strain of Escherichia coli ATCC 35218 (TEM-1, non-ESBL). Three experiments were run for 1 hour, 2 hours, 3 hours, 4 hours, 5 hours, and 6 hours and data was recorded related to the amount of the test strain that remained in the sample. The graphs shown in FIGS. 26B-26D illustrate the amount of the test strain in the sample versus time of exposure to blue light. The graph and data in FIGS. 26E-26F illustrate the overall findings of an aggregate of the experimental data shown in FIGS. 26B-26D, showing a reduction in the test strain due to blue light exposure.

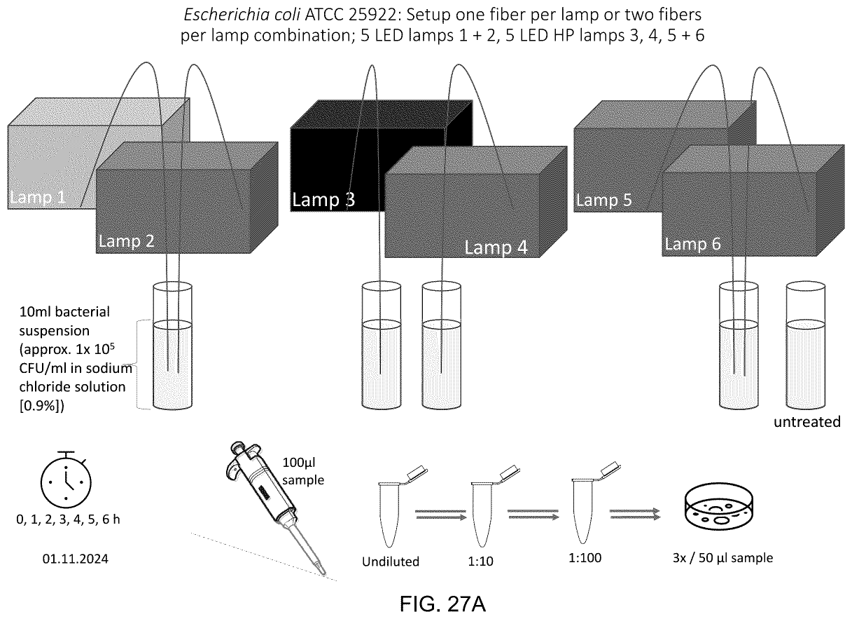

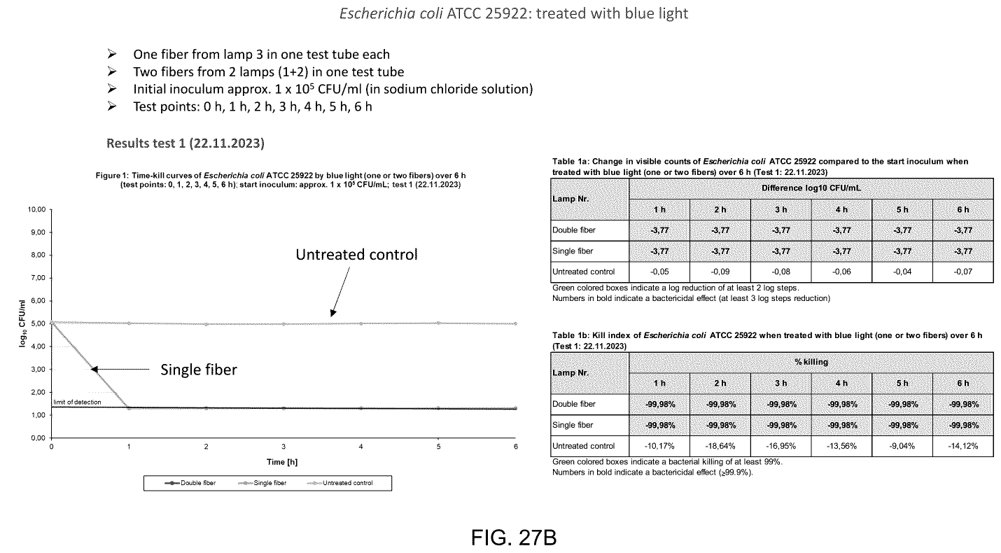

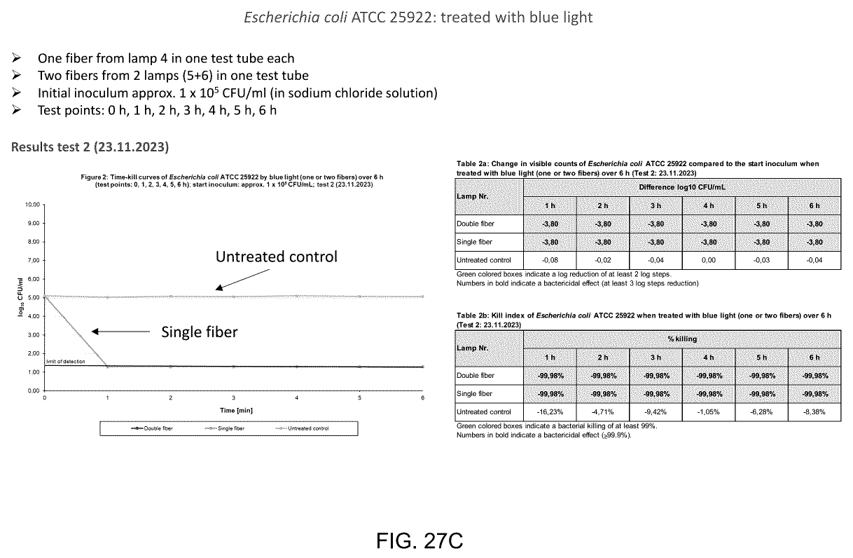

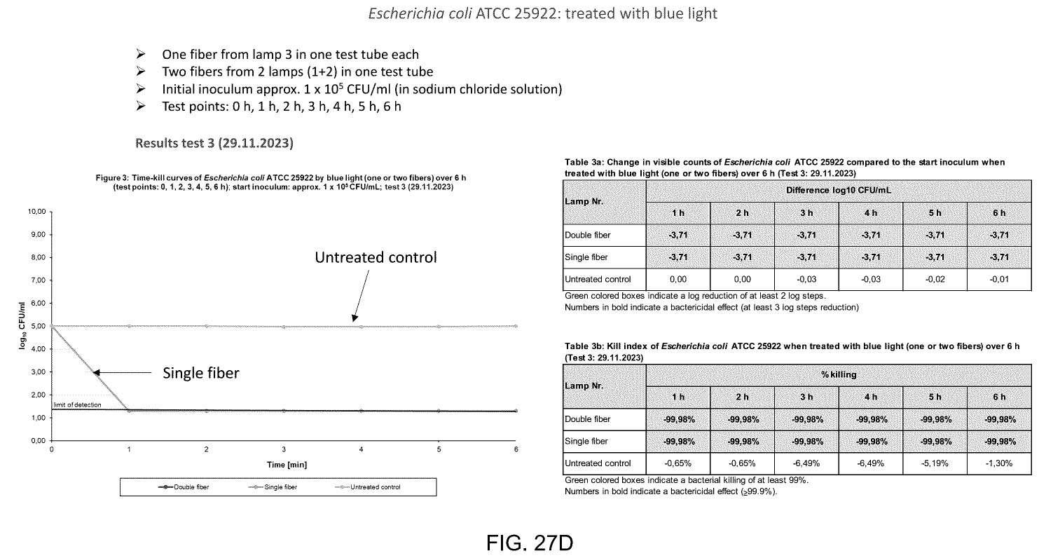

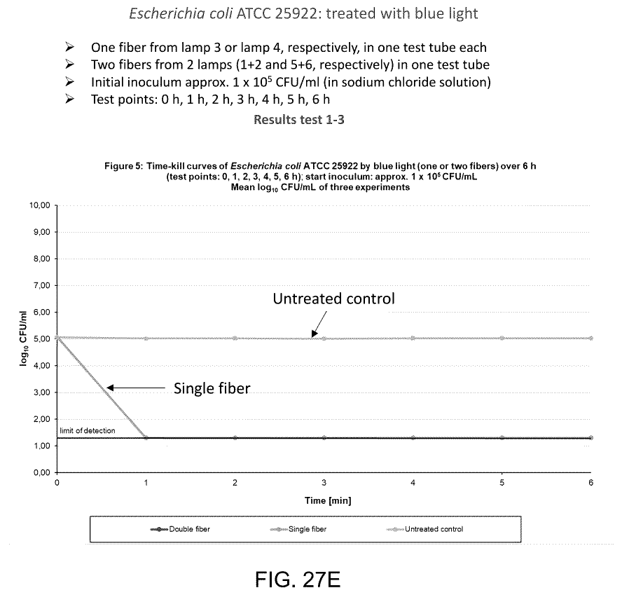

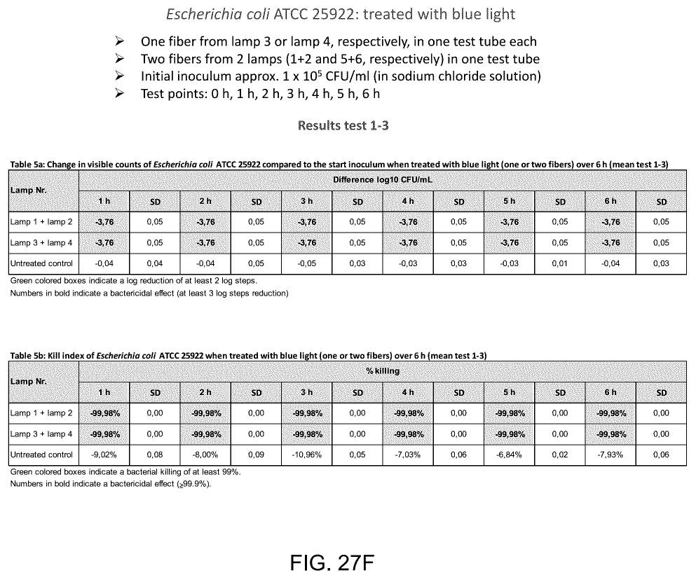

The test set up and results shown in FIGS. 27A-27F are for a test strain of Escherichia coli ATCC 25922. Three experiments were run for 1 hour, 2 hours, 3 hours, 4 hours, 5 hours, and 6 hours and data was recorded related to the amount of the test strain that remained in the sample. The graphs shown in FIGS. 27B-27D illustrate the amount of the test strain in the sample versus time of exposure to blue light. The graph and data in FIGS. 27E-27F illustrate the overall findings of an aggregate of the experimental data shown in FIGS. 27B-27D, showing a reduction in the test strain due to blue light exposure.

All patents, patent applications, and published references cited herein are hereby incorporated by reference in their entirety. It will be appreciated that several of the above-disclosed and other features and functions, or alternatives thereof, may be desirably combined into many other different systems or applications.

What is claimed is:

1. A device, comprising:
a main body comprising a first leg configured to engage a catheter, a second leg configured to engage a drainage tube, and a third leg configured to receive one or more optical fibers; and
a port coupled to the third leg, the port being configured to provide access for the one or more optical fibers and provide a seal relative to the one or more optical fibers, wherein the one or more optical fibers are configured to disperse light energy in the catheter such that intensity of the light energy dispersed in the catheter is distributed evenly over a length of the one or more optical fibers in both longitudinal and circumferential directions.

2. The device of claim 1, further comprising an anti-reflux valve configured to prevent fluid from flowing in a wrong direction out of the catheter.

3. The device of claim 1, further comprising a stop to hold the one or more optical fibers in position relative to the main body.

4. The device of claim 1, further comprising a compressive member configured to engage the catheter to increase a force of the seal around the catheter to increase a sterile barrier around the catheter.

5. The device of claim 4, wherein the compressive member is configured to apply a circumferential force to engage the catheter with the main body.

6. The device of claim 1, further comprising a strain relief configured to support the one or more optical fibers for a distance to prevent the one or more optical fibers from deforming during use.

7. The device of claim 1, wherein the catheter is a Foley catheter.

8. The device of claim 1, wherein the one or more optical fibers are configured to disperse light energy to treat pathogens associated with urinary tract infection.

9. The device of claim 1, wherein the one or more optical fibers include a cladding covering an outer surface thereof, and wherein at least a portion of the cladding of the one or more optical fibers is removed from an outer surface of the one or more optical fibers to achieve an even dispersion of the light energy.

10. The device of claim 1, wherein the one or more optical fibers sized to pass through an inner lumen of the catheter and being configured to deliver light energy to provide an antimicrobial effect to the tissue.

11. A system, comprising:
a catheter having an elongated shaft and an inner lumen therethrough;
a main body comprising a first leg configured to engage the catheter, a second leg configured to engage a drainage tube, and a third leg configured to receive one or more optical fibers; and
the one or more optical fibers sized to pass through the third leg of the main body into the inner lumen of the delivery catheter and being configured to deliver light energy to provide an antimicrobial effect to the tissue, the one or more optical fibers being configured to disperse the light energy such that an intensity of the light energy is distributed evenly over a length of the one or more optical fibers in both longitudinal and circumferential directions;
wherein the antimicrobial effect of the light energy is configured to affect bacteria.

12. The system of claim 11, further comprising a port coupled to the third leg, the port being configured to provide access for the one or more optical fibers and provide a seal relative to the one or more optical fibers.

13. The system of claim 11, wherein the one or more optical fibers include a cladding covering an outer surface thereof, and wherein at least a portion of the cladding of the one or more optical fibers is removed from an outer surface of the one or more optical fibers to achieve an even dispersion of the light energy.

14. The system of claim 13, wherein at least a portion of the cladding is removed to form a helical spiral along the length of the one or more optical fibers.

15. The system of claim 14, wherein the helical spiral becoming increasingly tight as the helical spiral moves from a proximal end of the one or more optical fibers to a distal end of the one or more optical fibers to achieve an even light distribution over the length of the one or more optical fibers.

16. The system of claim 11, wherein the one or more optical fibers includes a diffusive membrane disposed on an outer surface thereof, the diffusive membrane configured to be applied to the outer surface of the one or more optical fibers to achieve the even light distribution over the length of the one or more optical fibers.

17. The system of claim 11, wherein the light energy has illumination wavelengths from about 400 nm to about 475 nm.

18. The system of claim 11, wherein the light energy has illumination wavelengths from about 380 nm to about 500 nm.

19. The system of claim 11, wherein the light energy has illumination wavelengths from about 405 nm to about 470 nm.

20. A method, comprising:
engaging a catheter with a fitting, the fitting comprising a first leg configured to engage the catheter, a second leg configured to engage a drainage tube, and a third leg configured to receive one or more optical fibers;
receiving, by the third leg of the fitting, the one or more optical fibers;
passing the one or more optical fibers from the third leg of fitting into an inner lumen of the catheter; and
delivering light energy from the one or more optical fibers to the catheter to provide an antimicrobial effect, the one or more optical fibers being configured to disperse the light energy such that an intensity of the light energy is distributed evenly over a length of the one or more optical fibers in both longitudinal and circumferential directions.
